(19) United States
(12) Patent Application Publication
(10) Pub. No.: US 2008/0199416 A1
(43) Pub. Date: Aug. 21, 2008

(54) Use of Anionically and Cationically Ampholytic Copolymers

(75) Inventors: Son Nguyen Kim, Hemsbach (DE); Matthias Laubender, Schifferstadt (DE); Peter Hossel, Schifferstadt (DE)

Correspondence Address: CONNOLLY BOVE LODGE & HUTZ LLP, 1875 EYE STREET, N.W., SUITE 1100, WASHINGTON, DC, 20036, US

(73) Assignee: BASF AKTIENGESELLSCHAFT, Ludwigshafen (DE)

(21) Appl. No.: 11/996,440

(22) PCT Filed: Jul. 21, 2006
(86) PCT No.: PCT/EP06/64506

§ 371 Date: Jan. 22, 2008

(30) Foreign Application Priority Data
Jul. 22, 2005 (DE) 102005034412.7
Jul. 26, 2005 (DE) 102005034906.4
Sep. 30, 2005 (DE) 102005046916.7
Sep. 30, 2005 (DE) 102005046918.3

(51) Int. Cl. A61K 8/81; C08F 220/18; C08F 226/06; A61Q 5/00; C08F 220/60

(52) U.S. Cl. 424/70.11; 526/329.5; 526/258; 526/307; 526/312; 526/264

(57) ABSTRACT

The present invention relates to the use of an anionically ampholytic or cationically ampholytic copolymer which which is obtainable by free-radical copolymerization of
a1) at least one compound with a free-radically polymerizable, α,β-ethylenically unsaturated double bond and at least one anionogenic and/or anionic group per molecule,
a2) at least one compound with a free-radically polymerizable, α,β-ethylenically unsaturated double bond and at least one cationogenic and/or cationic group per molecule,
b) at least one free-radically polymerizable crosslinking compound which comprises at least two α,β-ethylenically unsaturated double bonds per molecule,
c) if appropriate in the presence of at least one silicone compound comprising a polyether group and/or a free-radically polymerizable olefinically unsaturated double bond,
   as rheology modifier for hair cosmetic compositions.

The following invention relates to the use of anionically ampholytic and cationically ampholytic copolymers as rheology modifiers for hair cosmetic compositions.

Specific requirements are often placed on hair cosmetic compositions with regard to their rheological properties. They can often only be converted to the desired application form using additives, so-called “thickeners”. Examples of customary low-molecular weight thickeners are, for example, the alkali metal and aluminum salts of fatty acids, fatty alcohols or waxes. However, depending on the field of use of the preparation to be thickened, use of the known thickeners is often associated with disadvantages. For example, the thickening effect of the thickeners may not be satisfactory, their use may be undesired or their incorporation into the preparation to be thickened may be hindered or completely impossible, for example due to their incompatibility with the compound to be thickened. The provision of products with a complex profile of properties using the lowest possible fraction or the fewest possible different active substances often presents difficulties. Thus, for example, there is a need for polymers for hair cosmetic compositions which allow the rheological properties of the compositions to be adjusted and which additionally have good film-forming properties. In addition, esthetic requirements are increasingly being placed on hair cosmetic products by the consumer. Thus, with such products, a preference for clear, opaque formulations in the form of gels is currently observed. A further advantageous property of polymers for hair cosmetic formulations is the ability to be converted into a solid form, in particular a powder and nevertheless be capable of being incorporated into a composition to be thickened within a short time and thereby reliably provide the desired Theological properties.

WO 01/85821 describes polyurethanes and their use for modifying rheological properties.

U.S. Pat. No. 3,915,921 describes copolymers which comprise, in copolymerized form, an olefinically unsaturated carboxylic acid, a C₁₀-C₃₀-alkyl(meth)acrylate and, if appropriate, a crosslinking monomer with at least two ethylenically unsaturated double bonds. In neutralized form, they serve as thickeners for diverse applications.

WO 97/21744 describes crosslinked anionic copolymers and their use as thickeners and dispersants in aqueous systems.

EP-A-0 982 021 describes the use of (partially) neutralized copolymers of

A) 50 to 99% by weight of monoethylenically unsaturated carboxylic acids and

B) 1 to 50% by weight of at least one comonomer chosen from
a) monoethylenically unsaturated carboxylic esters with saturated C₈-C₃₀-alcohols,
b) N-C₈-C₁₈-alkyl- and N,N-di-C₈-C₁₈-alkylcarboxamides,
c) vinyl esters of aliphatic C₈-C₃₀-carboxylic acids,
d) C₈-C₁₈-alkyl vinyl ethers,
and mixtures thereof
   as thickeners for producing hair-washing compositions.

U.S. Pat. No. 4,395,524 and U.S. Pat. No. 4,432,881 describe copolymers based on monomers containing amide groups which act as thickeners.

DE-A-42 13 971 describes copolymers which comprise, in copolymerized form, at least one olefinically unsaturated monomer containing acid groups, at least one olefinically unsaturated quaternary ammonium compound, if appropriate at least one polyether(meth)acrylate and, if appropriate, at least one crosslinker and their use as thickeners for thickening aqueous systems, which may be cosmetic preparations.

EP-A-893 117 and EP-A-913 143 describe crosslinked cationic copolymers and their use, inter alia, as hair-setting gel formers in cosmetic compositions.

EP-A-1 064 924 describes the use of crosslinked cationic polymers in skin cosmetic and dermatological preparations, inter alia as thickeners.

U.S. Pat. No. 5,015,708 describes a process for the preparation of a terpolymer comprising (i) a vinyllactam, (ii) a monomer containing acid groups and (iii) a hydrophobic monomer, which may, inter alia, be an ethylenically unsaturated silicone compound, by precipitation polymerization, and also the preparation of powders from these polymers.

WO 01/62809 describes a cosmetic composition which comprises at least one water-soluble or water-dispersible polymer that comprises, in incorporated form,
a) 5 to 50% by weight of at least one α,β-ethylenically unsaturated monomer with a tert-butyl group,
b) 25 to 90% by weight of at least one N-vinylamide and/or N-vinyllactam,
c) 0.5 to 30% by weight of at least one compound with a free-radically polymerizable, α,β-ethylenically unsaturated double bond and at least one cationogenic and/or cationic group per molecule, and
d) 0 to 30% by weight of at least one further α,β-ethylenically unsaturated compound, which may be compounds with at least one anionogenic and/or anionic group per molecule.

EP-A-670 333 describes crosslinked water-soluble polymer dispersions which are obtainable by polymerization of a monomer mixture comprising at least one water-soluble monomer, at least one crosslinker, and, if appropriate, hydrophobic and/or amphiphilic monomers in the presence of a polymeric dispersant. Besides a large number of others, water-soluble monomers which can be used are also N-vinylpyrrolidone, and monomers with cationic/cationizable groups, such as N-vinylimidazole. The polymeric dispersants may be polyelectrolytes which comprise, for example, copolymerized salts of (meth)acrylic acid as anionic monomer building blocks or quaternized derivatives of N,N-dimethylaminoethyl(meth)acrylate as cationic building blocks. A use of the polymer dispersions in cosmetics is not described.

EP-A-929 285 teaches the use of water-soluble copolymers which comprise vinylcarboxamide units and vinylimidazole units in copolymerized form as a constituent of cosmetic compositions. Polyelectrolyte complexes of these copolymers with polymers containing carboxylic acid groups are not disclosed in this document.

WO 00/27893 describes aqueous polymer dispersions based on N-vinylcarboxamides and, if appropriate, comonomers, in which case, besides a large number of others, mention is also made of N-vinylpyrrolidone, N-vinylimidazole and N-vinylimidazole derivates. The polymerization takes place in the presence of at least one polymeric dispersant. A use of these polymer dispersions in cosmetics is described only very generally and without being demonstrated by a working example.

EP-A-1038891 describes water-soluble or water-dispersible polymeric salts of at least one polymer and at least one oppositely charged neutralizing agent.

WO 02/41856 describes the use of polymer dispersions which are obtainable by polymerization of at least one water-soluble monomer in an aqueous salt solution which comprises at least one polyelectrolyte as dispersant for the cosmetic treatment of keratin materials. In addition, the dispersions comprise at least one agent for adjusting the viscosity, for example a polycarboxylic acid or a salt thereof. Water-soluble monomers which can be used are cationic, anionic and nonionic monomers, preference being given to monomer mixtures which comprise at least one cationic monomer and, if appropriate, in addition acrylic acid and/or acrylamide.

WO 2005/004821 describes a cosmetic or pharmaceutical composition which comprises at least one polyelectrolyte complex which comprises at least one water-soluble or water-dispersible copolymer A1) with cationogenic groups, which comprises, in copolymerized form, vinylimidazole and/or a derivative thereof and at least one further monomer copolymerizable therewith, and at least one polymer A2) containing acid groups.

WO 2005/005497 describes an aqueous polymer dispersion Pd) which is obtainable by free-radical polymerization of a monomer mixture M) comprising
a) at least one α,β-ethylenically unsaturated amide-group-containing compound of the general formula I

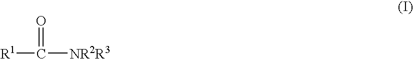

where
R² is a group of the formula CH₂═CR⁴— and R¹ and R³, independently of one another, are H, alkyl, cycloalkyl, heterocycloalkyl, aryl or hetaryl, or R¹ and R³ together with the amide group to which they are bonded are a lactam having 5 to 8 ring atoms,
b) at least one free-radically polymerizable crosslinking compound with at least two α,β-ethylenically unsaturated double bonds per molecule,
c) at least one compound with a free-radically polymerizable α,β-ethylenically unsaturated double bond and at least one cationogenic and/or cationic group per molecule,
   in an aqueous medium in the presence of at least one polymeric anionic dispersant D). Also described are cosmetic or pharmaceutical compositions which comprise such a polymer dispersion or a polymer obtainable by drying such a dispersion.

WO 00/39176 describes a hydrophilic, cationic, ampholytic copolymer which comprises, in copolymerized form, 0.05 to 20 mol % of an anionic monomer with at least one carboxy group, 0 to 45 mol % of a cationic monomer with at least one amino group and, if appropriate, a hydrophobic monomer and/or a crosslinker, where the molar ratio of cationic monomer to anionic monomer is about 2:1 to 16:1.

WO 04/058837 describes an ampholytic copolymer which is obtainable by free-radical copolymerization of
a) at least one ethylenically unsaturated compound with at least one anionogenic and/or anionic group,
b) at least one ethylenically unsaturated compound with at least one cationogenic and/or cationic group,
c) at least one unsaturated compound containing amide groups
   and if appropriate further comonomers. The polymerization can take place in the presence of a graft base, which may, inter alia, be a silicone derivative which contains polyalkylene oxide. Also described are polyelectrolyte complexes which comprise such an ampholytic copolymer, and cosmetic or pharmaceutical compositions based on these copolymers which contain silicone groups and polyelectrolyte complexes.

US 2006/0084586 A1 describes rheology-modifying hair-setting resins which comprise a crosslinked copolymer based on vinylamide and carboxylic acid monomers. Polymers which are obtainable by free-radical polymerization in the presence of at least one silicone compound having a polyether group and/or a free-radically polymerizable double bond are not described. Polymers which comprise at least one cationic compound which is chosen from N-vinylimidazole compounds, N-[3-(dimethylamino)-propyl]acrylamide, N-[3-(dimethylamino)propyl]methacrylamide and mixtures thereof in copolymerized form are likewise not described.

There is also a need for thickeners for hair cosmetic compositions with improved properties. Thus, the anionic thickeners based on acrylic acid, such as the corresponding carbopols, currently still used most often for these applications have a number of disadvantages. They are themselves not capable of film formation, meaning that, to prepare compositions with a setting effect, the use of relatively large amounts of film-forming polymers is always required. However, since anionic thickeners based on acrylic acid are incompatible with a large number of polyelectrolytes, suitable formulation partners are predominantly only nonionic polymers. Thus, a large number of products with interesting application properties, such as, for example, gels based on cationic polymers, cannot be prepared at all.

In particular, there is a need for polymeric thickeners for hair cosmetic compositions which are suitable for formulating gel-like preparations. These should combine as many of the following properties as possible:
the gels obtained should be as clear as possible,
they should be able to be distributed easily in the hair and nevertheless provide good hold, which can be achieved particularly well by gels with thixotropic properties,
they should themselves have film-forming properties and thus contribute to the setting of the hair,
they should have conditioning properties and improve the sensory properties of the hair, e.g. impart suppleness and shine to it and not be sticky, or only be sticky to a small degree, after drying,
the hair treated with the gel should have good wet combability (the freshly treated hair can thus be readily shaped using the comb in order to form the desired hairstyle),
ability to formulate gels in as many cosmetically acceptable pH ranges as possible, especially in a pH range of from about 3 to 9,
the ability to formulate gels with properties which can be switched in a targeted manner via the pH value.
   In addition, polymers for use as rheology modifiers for cosmetic compositions for treating hair should be able to be readily converted to a solid form, preferably a powder, which can be incorporated easily into the formulations to be thickened.

According to the invention, this object is achieved through the use of an ampholytic copolymer which has a molar excess of anionogenic/anionic groups compared with cationogenic/cationic groups or which has a molar excess of cationogenic/cationic groups compared with anionogenic/anionic groups and which is obtainable by free-radical copolymerization of
a1) at least one compound with a free-radically polymerizable, α,β-ethylenically unsaturated double bond and at least one anionogenic and/or anionic group per molecule,
a2) at least one compound with a free-radically polymerizable, α,β-ethylenically unsaturated double bond and at least one cationogenic and/or cationic group per molecule,
b) at least one free-radically polymerizable crosslinking compound which comprises at least two α,β-ethylenically unsaturated double bonds per molecule,
c) if appropriate in the presence of at least one silicone compound comprising a polyether group and/or a free-radically polymerizable olefinically unsaturated double bond,
   as rheology modifier for hair cosmetic compositions.

Copolymers which have a molar excess of anionogenic/anionic groups compared with cationogenic/cationic groups are also referred to as anionic ampholytic copolymers within the scope of the invention. Copolymers which have a molar excess of cationogenic/cationic groups compared with anionogenic/anionic groups are also referred to as cationic ampholytic copolymers within the scope of the invention.

In a first embodiment, copolymers free from silicone groups are used for the use according to the invention. Of suitability for this purpose are all of the copolymers described in the German patent application 10 2005 034 412.7 which have a molar excess of anionogenic/anionic groups compared with cationogenic/cationic groups or which have a molar excess of cationogenic/cationic groups compared with anionogenic/anionic groups. The disclosure of this document is hereby incorporated with regard to these copolymers in its entirety by reference.

In a second embodiment, copolymers containing silicone groups are used for the use according to the invention. Of suitability for this are all of the copolymers described in the German patent application 10 2005 034 906.4 which have a molar excess of anionogenic/anionic groups compared with cationogenic/cationic groups or which have a molar excess of cationogenic/cationic groups compared with anionogenic/anionic groups. The disclosure of this document with regard to these copolymers is hereby incorporated in its entirety by reference.

The use of an ampholytic copolymer which has a molar excess of anionogenic/anionic groups is described in the German priority application 10 2005 046 918.3. The use of an ampholytic copolymer which has a molar excess of cationogenic/cationic groups is described in the German priority application 10 2005 046 916.7. This disclosure is hereby incorporated in its entirety by reference.

The copolymers A) to be used according to the invention can be prepared by conventional polymerization processes, e.g. by solution polymerization or bulk polymerization. Copolymers with particularly advantageous properties, i.e. with generally higher molecular weights and a better ability to form pulverulent formulations than are obtained by conventional polymerization processes are obtained by the preparation in accordance with the method of precipitation polymerization. A preferred embodiment of the invention is therefore the use of copolymers which are obtainable by free-radical copolymerization by the method of precipitation polymerization in at least one organic solvent. In a specific embodiment, for the preparation of the copolymers used according to the invention, at least two free radical initiators are used whose decomposition temperatures and/or whose half-lives at a certain polymerization temperature are different from one another. Here, copolymers with particularly low residual monomer contents can be achieved. This is the case particularly if the initiator decomposing at the higher temperature is added before completion, preferably before the start of the precipitation of the polymer.

In the precipitation polymerization, the monomers used are soluble in the reaction medium (monomer, solvent), but the corresponding polymer is not. The polymer which forms becomes insoluble under the polymerization conditions chosen and precipitates out of the reaction mixture. In the process it is possible to obtain anionogenic/anionic ampholytic copolymers with molecular weights which are higher than those obtainable by other polymerization processes, e.g. by solution polymerization, which are particularly advantageously suitable as rheology modifiers (specifically thickeners).

For the purposes of the present invention, the expression alkyl comprises straight-chain and branched alkyl groups. Suitable short-chain alkyl groups are, for example, straight-chain or branched C₁-C₇-alkyl groups, preferably C₁-C₆-alkyl groups and particularly preferably C₁-C₄-alkyl groups. These include, in particular, methyl, ethyl, propyl, isopropyl, n-butyl, 2-butyl, sec-butyl, tert-butyl, n-pentyl, 1-methylbutyl, 2-methylbutyl, 3-methylbutyl, 1,2-dimethylpropyl, 1,1-dimethylpropyl, 2,2-dimethylpropyl, 1-ethylpropyl, n-hexyl, 1-methylpentyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl etc.

Suitable longer-chain C₈-C₃₀-alkyl groups and C₈-C₃₀-alkenyl groups are straight-chain and branched alkyl groups and alkenyl groups. Preference is given here to predominantly linear alkyl radicals as also occur in natural or synthetic fatty acids and fatty alcohols and in oxo alcohols, which may, if appropriate, additionally be mono-, di- or polyunsaturated. These include, for example, n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene), arachinyl(ene), behenyl(ene), lignocerinyl(ene), melissinyl(ene), etc.

Cycloalkyl is preferably C₅-C₈-cycloalkyl, such as cyclopentyl, cyclohexyl, cycloheptyl or cyclooctyl.

Aryl comprises unsubstituted and substituted aryl groups and is preferably phenyl, tolyl, xylyl, mesityl, naphthyl, fluorenyl, anthracenyl, phenanthrenyl, naphthacenyl and in particular phenyl, tolyl, xylyl or mesityl.

In the text below, compounds which are derived from acrylic acid and methacrylic acid may sometimes be referred to in short by adding the syllable “(meth)” to the compound derived from acrylic acid.

The copolymers used according to the invention can advantageously be formulated as gels under normal conditions (20° C.). “Gel-like consistency” is shown by formulations which have a higher viscosity than a liquid and which are self-supporting, i.e. which retain a shape imparted to them without a shape-stabilizing covering. In contrast to solid formulations, gel-like formulations can, however, easily be deformed under the application of shear forces. The viscosity of the gel-like compositions is preferably in a range from greater than 600 to about 60 000 mPas, particularly preferably from 6000 to 30 000 mPas. The gels are preferably hair gels.

For the purposes of the present invention, water-soluble monomers and polymers are understood as meaning monomers and polymers which dissolve in water at 20° C. in an amount of at least 1 g/l. Water-dispersible monomers and polymers are understood as meaning monomers and polymers which disintegrate into dispersible particles under the application of shear forces, for example by stirring. Hydrophilic monomers are preferably water-soluble or at least water-dispersible. The copolymers used according to the invention are generally water-soluble.

For the purposes of the present invention, “modification of rheological properties” is understood in the broad sense. Thus, the copolymers used according to the invention are generally suitable for thickening the consistency of liquid compounds within a wide range. Depending on the basic consistency of the liquid compound(s), flow properties from low viscosity to solid (no longer flowable) are generally achieved depending on the amount of copolymer used. “Modification of rheological properties” is therefore understood as meaning, inter alia, the increase in the viscosity of liquids, the improvement of the thixotropy properties of gels, the solidification of gels and waxes etc.

The ampholytic copolymers used according to the invention have both anionogenic and/or anionic groups and also cationogenic and/or cationic groups. For their preparation, the oppositely charged/chargeable monomers a1) and a2) can be used together, i.e. in the form of a monomer pair (“monomer salt”). In this monomer composition, the molar ratio of anionogenic and anionic groups of component a1) to cationogenic and cationic groups of component a2) is about 1:1 (i.e. monovalent monomers are essentially used in equimolar amounts). The monomer pairs can be prepared separately prior to being used for the polymerization. However, preference is given to the “in situ” preparation of the monomer pairs through joint use (e.g. joint feed) during the preparation of the copolymers.

Preferably, the fraction in the monomer pair of the compounds used for the polymerization is at least 1% by weight, preferably at least 2% by weight, in particular at least 3% by weight.

Preferably, the copolymers have a significant excess of anionogenic/anionic groups compared with cationogenic/cationic groups. In a specific embodiment, monomers with ionogenic or ionic groups are used for the copolymerization in amounts such that the copolymer has a molar excess of anionogenic/anionic groups compared with cationogenic/cationic groups or a molar excess of cationogenic/cationic groups compared with anionogenic/anionic groups of at least 1.2:1, particularly preferably at least 1.4:1, in particular at least 2:1, specifically at least 2.5:1.

Monomer a1)

The copolymers used according to the invention comprise, as compound a1), at least one compound with a free-radically polymerizable α,β-ethylenically unsaturated double bond and with at least one anionogenic and/or anionic group per molecule in copolymerized form. The component a1) can be used in an amount of from 0.1 to 96% by weight, such as, for example, 0.5 to 90% by weight, based on the total weight of the compounds used for the polymerization. In the case of anionically ampholytic copolymers the component a1) is preferably used in an amount of from 2 to 70% by weight, preferably 3 to 60% by weight, based on the total weight of the compounds used for the polymerization (i.e. components a1), a2), b), c) and, if present, d) to g)). In the case of cationically ampholytic copolymers the component a1) is preferably used in an amount of from 0.2 to 35% by weight, preferably 0.5 to 25% by weight, based on the total weight of the compounds used for the polymerization (i.e. components a1), a2), b), c) and, if present, d) to g)).

Preferably, the compounds a1) are chosen from monoethylenically unsaturated carboxylic acids, sulfonic acids, phosphonic acids and mixtures thereof.

The monomers a1) include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The monomers a1) also include the half-esters of monoethylenically unsaturated dicarboxylic acids having 4 to 10, preferably 4 to 6, carbon atoms, e.g. of maleic acid, such as monomethyl maleate. The monomers a1) also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosphonic acid. The monomers a1) also include the salts of the abovementioned acids, in particular the sodium, potassium and ammonium salts, and the salts with amines. The monomers a1) can be used as they are or as mixtures with one another. The weight fractions given all refer to the acid form.

Preferably, the component a1) is chosen from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid, 2-acrylamido-2-methylpropanesulfonic acid, vinylphosphonic acid and mixtures thereof.

The component a1) is particularly preferably chosen from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof.

The component a1) is particularly preferably chosen from acrylic acid, methacrylic acid and mixtures thereof. In a specific embodiment, the component a1) comprises methacrylic acid or consists thereof. In another specific embodiment, the component a1) comprises acrylic acid or consists thereof.

Monomer a2)

The copolymers used according to the invention comprise at least one compound a2) with at least one cationogenic and/or cationic group per molecule in copolymerized form. The component a2) can generally be used in an amount of from 1 to 96% by weight, such as, for example, 2 to 90% by weight, based on the total weight of the compounds used for the polymerization. The anionically ampholytic copolymers comprise preferably 2 to 35% by weight, particularly preferably 3 to 30% by weight, based on the total weight of the monomers used for the polymerization, of at least one monomer a2) in copolymerized form. The cationically ampholytic copolymers preferably comprise 2 to 90% by weight, particularly preferably 3 to 70% by weight, in particular 5 to 70% by weight, based on the total weight of the monomers used for the polymerization, of at least one monomer a2) in copolymerized form.

Preferably, the component a2) comprises at least one compound which is chosen from esters of α,β-ethylenically unsaturated mono- and dicarboxylic acids with amino alcohols which may be mono- or dialkylated on the amine nitrogen, amides of α,β-ethylenically unsaturated mono- and dicarboxylic acids with diamines which have at least one primary or secondary amino group, N,N-diallylamine, N,N-diallyl-N-alkylamines and derivatives thereof, vinyl- and allyl-substituted nitrogen heterocycles, vinyl- and allyl-substituted heteroaromatic compounds and mixtures thereof.

In a preferred embodiment, the component a2) comprises, as vinyl-substituted heteroaromatic compound, at least one N-vinylimidazole compound. In a specific embodiment, the component a2) is chosen from N-vinylimidazole compounds and mixtures which comprise at least one N-vinylimidazole compound.

Preferably, the cationogenic and/or cationic groups of the component a2) are nitrogen-containing groups, such as primary, secondary and tertiary amino groups, and quaternary ammonium groups. The nitrogen-containing groups are preferably tertiary amino groups or quaternary ammonium groups. Charged cationic groups can be produced from the amine nitrogens either by protonation or by quaternization with acids or alkylating agents. These include, for example, carboxylic acids, such as lactic acid, tartaric acid or citric acid, or mineral acids, such as phosphoric acid, sulfuric acid and hydrochloric acid, or as alkylating agents C₁-C₄-alkyl halides or sulfates, such as ethyl chloride, ethyl bromide, methyl chloride, methyl bromide, dimethyl sulfate and diethyl sulfate. A protonation or quaternization can generally take place either before or after the polymerization, preferably after the polymerization.

Suitable N-vinylimidazole compounds a2) are compounds of the formula

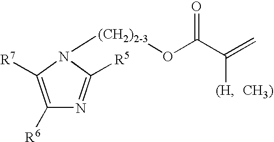

in which R⁵ to R⁷, independently of one another, are hydrogen, C₁-C₄-alkyl or phenyl. Preferably, R⁵ to R⁷ are hydrogen.

Furthermore, the copolymer preferably comprises, as monomer a2), at least one N-vinylimidazole compound of the general formula (II)

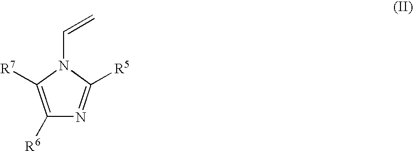

in copolymerized form, in which R⁵ to R⁷, independently of one another, are hydrogen, C₁-C₄-alkyl or phenyl.

Examples of compounds of the general formula (II) are given in Table 1 below:

TABLE 1

| R⁵ | R⁶ | R⁷ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

As monomer a2), preference is given to 1-vinylimidazole (N-vinylimidazole) and mixtures which comprise N-vinylimidazole.

Suitable monomers a2) are also the compounds obtainable by protonation or quaternization of the abovementioned N-vinylimidazole compounds. Examples of such charged monomers a2) are quaternized vinylimidazoles, in particular 3-methyl-1-vinylimidazolium chloride and methosulfate. Suitable acids and alkylating agents are those listed above.

In a specific embodiment, instead of or in addition to the abovementioned N-vinylimidazole compounds, the copolymers can comprise at least one other monomer a2) with at least one cationogenic and/or cationic groups in copolymerized form. In general, the fraction of these monomers a2) is 0 to 60% by weight, such as, for example, 0.1 to 50% by weight, based on the total weight of the compounds used for the polymerization. Preferably, the fraction of these monomers a2) in the case of anionically ampholytic copolymers is 0 to 30% by weight, particularly preferably 0.1 to 20% by weight, in particular 0.5 to 15% by weight, based on the total weight of the compounds used for the polymerization. Preferably, in the case of cationically ampholytic copolymers, the fraction of these monomers a2) is 0 to 30% by weight, particularly preferably 0.1 to 20% by weight, in particular 0.5 to 15% by weight, based on the total weight of the compounds used for the polymerization.

If at least one N-vinylimidazole compound, specifically N-vinylimidazole, is used as sole monomer a2), then the fraction is preferably 3 to 96% by weight, based on the total weight of the compounds used for the polymerization.

Suitable compounds a2) are the esters of α,β-ethylenically unsaturated mono- and dicarboxylic acids with amino alcohols. Preferred amino alcohols are C₂-C₁₂-amino alcohols which are C₁-C₈-mono- or -dialkylated on the amine nitrogen. Suitable acid components of these esters are, for example, acrylic acid, methacrylic acid, fumaric acid, maleic acid, itaconic acid, crotonic acid, maleic anhydride, monobutyl maleate and mixtures thereof. As acid component, preference is given to using acrylic acid, methacrylic acid and mixtures thereof.

Preferred monomers a2) are N-tert-butylaminoethyl(meth)acrylate, N,N-dimethylaminomethyl(meth)acrylate, N,N-dimethylaminoethyl(meth)acrylate, N,N-diethylaminoethyl(meth)acrylate, N,N-dimethylaminopropyl(meth)acrylate, N,N-diethylaminopropyl(meth)acrylate and N,N-dimethylaminocyclohexyl(meth)acrylate. Particular preference is given to N-tert-butylaminoethyl(meth)acrylate and N,N-dimethylaminoethyl(meth)acrylate. Particular preference is furthermore given to N,N-dimethylaminoethyl acrylate and N,N-dimethylaminoethyl methacrylate.

Suitable monomers a2) are also the amides of the abovementioned α,β-ethylenically unsaturated mono- and dicarboxylic acids with diamines which have at least one primary or secondary amino group. Preference is given to diamines which have one tertiary and one primary or secondary amino group.

Preferred monomers a2) are, for example, N-tert-butylaminoethyl(meth)acrylamide, N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexyl]acrylamide and N-[4-(dimethylamino)cyclohexyl]methacrylamide. Particular preference is given to N-[3-(dimethylamino)propyl]acrylamide, N-[3-(dimethylamino)propyl]methacrylamide (DMAPMAM) and mixtures thereof.

A specific embodiment relates to copolymers A) which comprise N-[3-(dimethylamino)propyl]acrylamide and/or N-[3-(dimethylamino)propyl]methacrylamide and no vinylimidazole compound. In a very specific embodiment, the component a2) consists only of N-[3-(dimethylamino)propyl]acrylamide and/or N-[3-(dimethylamino)propyl]methacrylamide. The fraction of N-[3-(dimethylamino)propyl]acrylamide and N-[3-(dimethylamino)propyl]methacrylamide (in total if both are present) is then preferably 2 to 95% by weight, particularly preferably 3 to 60% by weight, based on the total weight of the monomers used for the polymerization.

Suitable monomers a2) are also N,N-diallylamines and N,N-diallyl-N-alkylamines and acid addition salts thereof and quaternization products. Alkyl here is preferably C₁-C₂₄-alkyl. Preference is given to N,N-diallyl-N-methylamine and N,N-diallyl-N,N-dimethylammonium compounds, such as, for example, the chlorides and bromides. Particular preference is given to N,N-diallyl-N-methylamine.

Suitable monomers a2) are also vinyl- and allyl-substituted nitrogen heterocycles different from vinylimidazoles, such as 2- and 4-vinylpyridine, 2- and 4-allylpyridine, and the salts thereof.

Crosslinker b)

The copolymers can, if desired, comprise at least one crosslinker, i.e. a compound with two or more than two ethylenically unsaturated, nonconjugated double bonds in copolymerized form.

Preferably, crosslinkers are used in an amount of from 0.01 to 5% by weight, particularly preferably 0.1 to 4% by weight, based on the total weight of the monomers used for the polymerization.

Suitable crosslinkers b) are, for example, acrylic esters, methacrylic esters, allyl ethers or vinyl ethers of at least dihydric alcohols. The OH groups of the parent alcohols here may be completely or partially etherified or esterified; however, the crosslinkers comprise at least two ethylenically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000. Apart from the homopolymers of ethylene oxide and propylene oxide it is also possible to use block copolymers of ethylene oxide or propylene oxide or copolymers which comprise incorporated ethylene oxide and propylene oxide groups. Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. The polyhydric alcohols can of course also be used following reaction with ethylene oxide or propylene oxide as the corresponding ethoxylates or propoxylates. The polyhydric alcohols can also firstly be converted into the corresponding glycidyl ethers by reaction with epichlorohydrin. Preference is given to ethylene glycol di(meth)acrylate and polyethylene glycol di(meth)acrylates.

Further suitable crosslinkers b) are the vinyl esters or the esters of monohydric, unsaturated alcohols with ethylenically unsaturated C₃-C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. However, it is also possible to esterify the mono-hydric, unsaturated alcohols with polybasic carboxylic acids, for example malonic acid, tartaric acid, trimellitic acid, phthalic acid, terephthalic acid, citric acid or succinic acid.

Further suitable crosslinkers b) are esters of unsaturated carboxylic acids with the above-described polyhydric alcohols, for example of oleic acid, crotonic acid, cinnamic acid or 10-undecenoic acid.

Suitable crosslinkers b) are also straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Also suitable as crosslinkers b) are the acrylamides, methacrylamides and N-allylamines of at least difunctional amines. Such amines are, for example, 1,1-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacrylic acid, itaconic acid, maleic acid, or at least dibasic carboxylic acids as have been described above.

In addition, triallylamine and triallylmonoalkylammonium salts, e.g. triallylmethylammonium chloride or methyl sulfate, are suitable as crosslinker b).

Also suitable are N-vinyl compounds of urea derivatives, at least difunctional amides, cyanurates or urethanes, for example of urea, ethyleneurea, propyleneurea or tartardiamide, e.g. N,N′-divinylethyleneurea or N,N′-divinylpropyleneurea.

Further suitable crosslinkers b) are divinyldioxane, tetraallylsilane or tetravinylsilane.

It is of course also possible to use mixtures of the abovementioned crosslinkers b).

As crosslinkers b), very particular preference is given to ethylene glycol di(meth)acrylate, polyethylene glycol di(meth)acrylates, pentaerythritol triallyl ether, methylenebisacrylamide, N,N′-divinylethyleneurea, triallylamine and triallylmonoalkylammonium salts.

Silicone Compound c)

In the first embodiment described at the beginning, copolymers which are free from silicone groups are used. Of suitability for this, as already mentioned, are all of the copolymers described in the German patent application 10 2005 034 412.7. In the second embodiment of the invention described at the beginning, copolymers containing silicone groups are used. As already listed, of suitability for this are all of the copolymers described in the German patent application 10 2005 034 906.4, which have a molar excess of anionogenic/anionic groups compared with cationogenic/cationic groups.

The copolymers containing silicone groups used according to the invention are prepared by polymerization together with at least one silicone compound. The amount of silicone compound c) used is preferably 0.05 to 30% by weight, particularly preferably 0.1 to 20% by weight, in particular 0.2 to 10% by weight, specifically 0.5 to 5% by weight, based on the total weight of the compounds used for the polymerization.

Suitable silicone compounds c) are either compounds which have a free-radically polymerizable olefinically unsaturated double bond, or compounds which have a polyether group instead of such a double bond. Also suitable are of course compounds which have both at least one free-radically polymerizable double bond and also a polyether group.

In a first embodiment, the free-radical copolymerization to prepare the copolymers containing silicone groups takes place in the presence of at least one polyether-containing silicone compound c) which does not comprise α,β-ethylenically unsaturated double bonds. The copolymerization then takes place, in particular, in accordance with the method of precipitation polymerization in at least one organic solvent. The polymerization temperature is then preferably at at least 70° C., in particular preferably at least 80° C. Free-radical copolymerization in the presence of such a component c) produces copolymers with advantageous properties. This can be attributed, for example, to the effect of the component c) as protective colloid or emulsifier. This can, for example, also result from an at least partial grafting onto the component c) as graft base. However, mechanisms other than grafting are also conceivable. The copolymers containing silicone groups according to the invention comprise, quite generally, the process products of the free-radical copolymerization, which is understood as meaning, for example, pure graft polymers, mixtures of graft polymers with ungrafted compounds of the component c), copolymers of the abovementioned monomers, and any mixtures.

Suitable silicone derivatives c) are compounds known under the INCI names dimethicone copolyols or silicone surfactants, such as, for example, the compounds available under the trade names Abil® (from Th. Goldschmidt), Alkasil® (from Rhône-Poulenc), Silicone Polyol Copolymer® (from Genesee), Belsil® (from Wacker), Silwet® (from OSI) or Dow Corning (from Dow Corning). These include compounds with the CAS numbers 64365-23-7; 68937-54-2; 68938-54-5; 68937-55-3. A suitable commercially available compound is Belsil® DMC 6031.

Particularly suitable compounds c) are those which comprise the following structural elements:

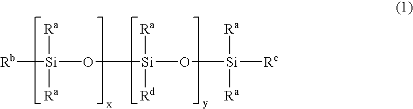

where:

the radicals R^{a} may be identical or different, and are chosen from alkyl, cycloalkyl, cycloalkylalkyl, aryl and arylalkyl, in particular from C₁-C₈-alkyl, C₅-C₈-cycloalkyl, benzyl and phenyl,

R^{b}, R^{c} and R^{d}, independently of one another, have one of the meanings given above for R^{a} or are —(CH₂)₁₋₆—OH, —(CH₂)₁₋₆—NHR^{e} or a radical of the formula (1.1)

—(CH₂)₁₋₆—O—(CH₂CH₂O)ₐ(CH₂CH(CH₃)O)_{b}—(C═O)_{c}—R^{f} (1.1)

where
in the formula (1.1) the order of the alkylene oxide units is arbitrary,
a and b, independently of one another, are an integer from 0 to 200, where the sum of a and b is >0,
c is 0 or 1,
R^{e} is hydrogen, C₁-C₈-alkyl or C₅-C₈-cycloalkyl,
R^{f} is hydrogen, C₁-C₄₀-alkyl, preferably methyl, or if c=0, may be the anion of an inorganic acid,
   with the proviso that at least one of the radicals R^{b}, R^{c} and R^{d} is a radical of the formula (1.1).

Preferably, x and y are chosen so that the molecular weight of the polysiloxane block is between 300 and 30 000.

Preferably, the radical R^{d} is a radical of the formula (1.1).

Preferably, R^{b} and R^{c} are C₁-C₈-alkyl, in particular methyl.

Preferred radicals (1.1) are those in which the sum of a+b is between 5 and 200.

Preferably, the groups R^{a} are chosen from the following group: methyl, ethyl, propyl, butyl, isobutyl, pentyl, isopentyl, hexyl, octyl, decyl, dodecyl and octadecyl, cycloaliphatic radicals, specifically cyclohexyl, aromatic groups, specifically phenyl or naphthyl, mixed aromatic-aliphatic radicals, such as benzyl or phenylethyl, and tolyl, xylyl and radicals of the formula (1.1).

Particularly suitable radicals R^{f} are those in which if c=1, R^{f} is any alkyl, cycloalkyl or aryl radical which has between 1 and 40 carbon atoms and which can carry further ionogenic groups, such as NH₂, COOH, SO₃H.

Preferred inorganic radicals R^{f} are, if c=0, phosphate and sulfate.

Particularly preferred silicone derivatives c) are those of the general structure:

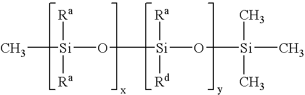

in which R^{d} is a radical of the formula (1.1)

—(CH₂)₁₋₆—O—(CH₂CH₂O)ₐ(CH₂CH(CH₃)O)_{b}—(C═O)_{c}—R^{f} (1.1)

as defined above. Specifically, it is Belsil® DMC 6031 from Wacker.

In a further embodiment, the silicone compound c) is an α,β-ethylenically unsaturated compound which has at least one polysiloxane group.

Suitable ethylenically unsaturated monomers c) with a polysiloxane group are, for example, compounds of the general formula V:

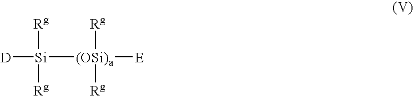

in which
D is an ethylenically unsaturated group which is preferably chosen from vinyl groups, vinyl(C₁-C₄-alkylene) groups, acryloyloxy(C₁-C₄-alkylene) groups and methacryloyloxy(C₁-C₄-alkylene) groups,
R^{g} is in each case, independently of one another, identical or different groups which are chosen from C₁-C₁₀-alkyl, phenyl, benzyl, C₄-C₈-cycloalkyl, and polyalkylene, polyoxyalkylene and polyalkyleneimine groups which can have a terminal alkyl ether, ester or amide function,
E can have the meanings given for R^{g} or those for D and
a is an integer from 1 to 1000, preferably 2 to 250.

Suitable compounds of the formula V are described, for example, in EP-A-0 408 311, which is hereby incorporated in its entirety by reference.

Further suitable silicone compounds c) which have at least one free-radically polymerizable double bond are free-radically polymerizable urethane(meth)acrylates which contain siloxane groups. Of suitability are, for example, the (meth)acrylate-functionalized organopolysiloxane-urethane copolymers described in EP-A-0 274 699 which are obtainable by reacting a polysiloxane functionalized with amino groups with urethane(meth)acrylate oligomers. The disclosure of this document is hereby incorporated by reference.

Preference is given to the compounds described in WO 2004/055088. As compound c) preference is also given to using at least one free-radically polymerizable urethane(meth)acrylate which contains siloxane groups, as described in WO 00/12588. These are urethane(meth)acrylates c) containing siloxane groups which comprise, in incorporated form,
α) at least one compound which comprises at least one active hydrogen atom and at least one free-radically polymerizable, α,β-ethylenically unsaturated double bond per molecule,
β) at least one diisocyanate,
γ) at least one compound which comprises two active hydrogen atoms per molecule,
δ) at least one compound which comprises at least one active hydrogen atom and at least one siloxane group per molecule,
   and the salts thereof.

For the purposes of the present invention, the expression “urethane(meth)acrylates” comprises, in quite general terms, compounds which have at least one olefinically unsaturated free-radically polymerizable double bond. These also include allylically unsaturated compounds. In addition, the expression “urethane(meth)acrylates” also comprises compounds which have urea groups instead of or in addition to the urethane groups. Urea groups result during the reaction of an isocyanate group with a primary or secondary amino group.

Component α)

Suitable compounds α) are, for example, the customary vinyl compounds known to the person skilled in the art which additionally have at least one group which is reactive toward isocyanate groups which is preferably chosen from hydroxyl groups and primary and secondary amino groups. These include, for example, the esters of α,β-ethylenically unsaturated mono- and dicarboxylic acids with at least dihydric alcohols. α,β-Ethylenically unsaturated mono- and/or dicarboxylic acids which may be used are, for example, acrylic acid, methacrylic acid, fumaric acid, maleic acid, crotonic acid, itaconic acid etc. and mixtures thereof. Suitable alcohols are customary diols, triols and polyols, e.g. 1,2-ethanediol, 1,3-propanediol, 1,4-butanediol, 1,5-pentanediol, 1,6-hexanediol, 1,10-decanediol, diethylene glycol, 2,2,4-trimethylpentanediol-1,5,2,2-dimethylpropanediol-1,3,1,4-dimethylolcyclohexane, 1,6-dimethylolcyclohexane, glycerol, trimethylolpropane, erythritol, pentaerythritol, sorbitol etc. The compounds a) are then, for example, hydroxymethyl(meth)acrylate, hydroxyethyl ethacrylate, 2-hydroxyethyl(meth)acrylate, 2-hydroxypropyl(meth)acrylate, 3-hydroxypropyl(meth)acrylate, 3-hydroxybutyl(meth)acrylate, 4-hydroxybutyl(meth)acrylate, 6-hydroxyhexyl(meth)acrylate, 3-hydroxy-2-ethylhexyl(meth)acrylate, and di(meth)acrylic esters of 1,1,1-trimethylolpropane or of glycerol.

Suitable monomers α) are also the esters and amides of the abovementioned α,β-ethylenically unsaturated mono- and dicarboxylic acids with C₂-C₁₂-amino alcohols which have a primary or secondary amino group. These include aminoalkyl acrylates and aminoalkyl methacrylates and their N-monoalkyl derivatives, which carry, for example, an N-C₁-C₈-monoalkyl radical, such as aminomethyl(meth)acrylate, aminoethyl(meth)acrylate, N-methylaminomethyl(meth)acrylate, N-ethylaminomethyl(meth)acrylate, N-ethylaminoethyl(meth)acrylate, N-(n-propyl)aminomethyl(meth)acrylate, N-isopropylaminomethyl(meth)acrylate and preferably tert-butylaminoethyl acrylate and tert-butylaminoethyl methacrylate. These also include N-(hydroxy-C₁-C₁₂-alkyl)(meth)acrylamides, such as N-hydroxymethyl(meth)acrylamide, N-hydroxyethyl(meth)acrylamide etc.

Suitable monomers α) are also the amides of the abovementioned α,β-ethylenically unsaturated mono- and dicarboxylic acids with di- and polyamines which have at least two primary or two secondary or one primary and one secondary amino group(s). These include, for example, the corresponding amides of acrylic acid and methacrylic acid, such as aminomethyl(meth)acrylamide, aminoethyl(meth)acrylamide, aminopropyl(meth)acrylamide, amino-n-butyl(meth)acrylamide, methylaminoethyl(meth)acrylamide, ethylaminoethyl(meth)acrylamide, methylaminopropyl(meth)acrylamide, ethylaminopropyl(meth)acrylamide, methylamino-n-butyl(meth)acrylamide etc.

Suitable monomers a) are also the reaction products of epoxide compounds which have at least one epoxide group with the abovementioned α,β-ethylenically unsaturated mono- and/or dicarboxylic acids and anhydrides thereof. Suitable epoxide compounds are, for example, glycidyl ethers, such as bisphenol A diglycidyl ether, resorcinol diglycidyl ether, 1,3-propanediol diglycidyl ether, 1,4-butanediol diglycidyl ether, 1,5-pentanediol diglycidyl ether, 1,6-hexanediol diglycidyl ether etc.

Component β)

Component β) is a customary aliphatic, cycloaliphatic and/or aromatic diisocyanate, such as tetramethylene diisocyanate, hexamethylene diisocyanate, methylenediphenyl diisocyanate, 2,4- and 2,6-tolylene diisocyanate and isomer mixtures thereof, o- and m-xylylene diisocyanate, 1,5-naphthylene diisocyanate, 1,4-cyclohexylene diisocyanate, dicyclohexylmethane diisocyanate and mixtures thereof. Component β) is preferably hexamethylene diisocyanate, isophorone diisocyanate, o- and m-xylylene diisocyanate, dicyclohexylmethane diisocyanate and mixtures thereof. If desired, up to 3 mol % of the specified compounds can be replaced by triisocyanates.

Component γ)

Suitable compounds of component γ) are, for example, diols, diamines, amino alcohols and mixtures thereof. The molecular weight of these compounds is preferably in a range from about 56 to 280. If desired, up to 3 mol % of the specified compounds can be replaced by triols or triamines.

Suitable diols γ) are, for example, ethylene glycol, propylene glycol, butylene glycol, neopentyl glycol, cyclohexanedimethylol, di-, tri-, tetra-, penta- or hexaethylene glycol and mixtures thereof. Preference is given to using neopentyl glycol and/or cyclohexanedimethylol.

Suitable amino alcohols γ) are, for example, 2-aminoethanol, 2-(N-methylamino)ethanol, 3-aminopropanol, 4-aminobutanol, 1-ethylaminobutan-2-ol, 2-amino-2-methyl-1-propanol, 4-methyl-4-aminopentan-2-ol etc.

Suitable diamines γ) are, for example, ethylenediamine, propylenediamine, 1,4-diaminobutane, 1,5-diaminopentane and 1,6-diaminohexane.

Preferred compounds of the component γ) are polymers with a number-average molecular weight in the range from about 300 to 5000, preferably about 400 to 4000, in particular 500 to 3000. These include, for example, polyesterdiols, polyetherols, α,ω-diaminopolyethers and mixtures thereof. Preference is given to using polymers containing ether groups.

The polyetherols γ) are preferably polyalkylene glycols, e.g. polyethylene glycols, polypropylene glycols, polytetrahydrofurans etc., block copolymers of ethylene oxide and propylene oxide or block copolymers of ethylene oxide, propylene oxide and butylene oxide which comprise the copolymerized alkylene oxide units in random distribution or in the form of blocks.

Suitable α,ω-diaminopolyethers γ) can be prepared, for example, by amination of polyalkylene oxides with ammonia.

Suitable polytetrahydrofurans γ) can be prepared by cationic polymerization of tetrahydrofuran in the presence of acidic catalysts, such as, for example, sulfuric acid or fluorosulfuric acid. Such preparation processes are known to the person skilled in the art.

Polyesterdiols γ) which can be used preferably have a number-average molecular weight in the range from about 400 to 5000, preferably 500 to 3000, in particular 600 to 2000.

Suitable polyesterdiols are all those which are customarily used for the preparation of polyurethanes, in particular those based on aromatic dicarboxylic acids, such as terephthalic acid, isophthalic acid, phthalic acid, Na or K sulfoisophthalic acid etc., aliphatic dicarboxylic acids, such as adipic acid or succinic acid etc., and cycloaliphatic dicarboxylic acids, such as 1,2-, 1,3- or 1,4-cyclohexanedicarboxylic acid. Suitable diols are, in particular, aliphatic diols, such as ethylene glycol, propylene glycol, 1,6-hexanediol, neopentyl glycol, diethylene glycol, polyethylene glycols, polypropylene glycols, 1,4-dimethylolcyclohexane, and poly(meth)acrylatediols of the formula

HO—C(—R′)(—COOR″)—OH

in which R′ is H or CH₃ and R″ is C₁-C₁₈-alkyl (in particular C₁-C₁₂- or C₁-C₈-alkyl) which have a molar mass of up to about 3000. Diols of this type can be prepared in the usual way and are commercially available (Tegomer® grades MD, BD and OD from Goldschmidt).

Preference is given to polyesterdiols based on aromatic and aliphatic dicarboxylic acids and aliphatic diols, in particular those in which the aromatic dicarboxylic acid constitutes 10 to 95 mol %, in particular 40 to 90 mol % and preferably 50 to 85 mol %, of the total dicarboxylic acid fraction (remainder aliphatic dicarboxylic acids).

Particularly preferred polyesterdiols are the reaction products of phthalic acid/diethylene glycol, isophthalic acid/1,4-butanediol, isophthalic acid/adipic acid/1,6-hexanediol, 5-NaSO₃-isophthalic acid/phthalic acid/adipic acid/1,6-hexanediol, adipic acid/ethylene glycol, isophthalic acid/adipic acid/neopentyl glycol, isophthalic acid/adipic acid/neopentyl glycol/diethylene glycol/dimethylolcyclohexane and 5-NaSO₃-isophthalic acid/isophthalic acid/adipic acid/neopentyl glycol/diethylene glycol/dimethylolcyclohexane.

The compounds of the component γ) can be used individually or as mixtures.

Component δ)

Preferably, the component δ) is chosen from:
polysiloxanes of the general formula VI.1

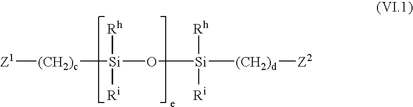

in which
c and d, independently of one another, are 2 to 8,
e is 3 to 100,
R^{h} and Rⁱ, independently of one another, are C₁-C₈-alkyl, benzyl or phenyl,
Z¹ and Z², independently of one another, are OH, NHR^{k} or a radical of the formula VII

—O—(CH₂CH₂O)ᵥ(CH₂CH(CH₃)O)_{w}—H (VII)
where
in the formula VII the order of the alkylene oxide units is arbitrary and v and w, independently of one another, are an integer from 0 to 200, where the sum of v and w is >0,
R^{k} is hydrogen, C₁-C₈-alkyl or C₅-C₈-cycloalkyl;
polysiloxanes of the general formula VI.2

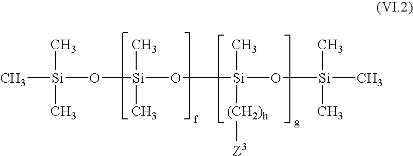

in which
the order of the siloxane units is arbitrary,
f and g, independently of one another, are 0 to 100, where the sum of f and g is at least 2,
h is an integer from 2 to 8,
Z³ is OH, NHR^{k} or a radical of the formula VII,
   where R^{k} is hydrogen, C₁-C₈-alkyl, C₅-C₈-cycloalkyl or a radical of the formula —(CH₂)ᵤ—NH₂, where u is an integer from 1 to 10, preferably 2 to 6,
polysiloxanes with repeat units of the general formula VI.3

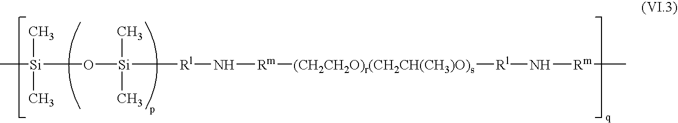

in which
p is an integer from 0 to 100,
q is an integer from 1 to 8,
R^{l} and R^{m}, independently of one another, are C₁-C₈-alkylene,
the order of the alkylene oxide units is arbitrary and
r and s, independently of one another, are an integer from 0 to 200, where the sum of r and s is >0,
polysiloxanes of the general formula VI.4

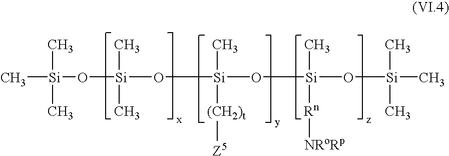

in which
Rⁿ is a C₁-C₈-alkylene radical,
R^{o} and R^{p}, independently of one another, are hydrogen, C₁-C₈-alkyl or C₅-C₈-cycloalkyl,
the order of the siloxane units is arbitrary,
x, y and z, independently of one another, are 0 to 100, where the sum of x, y and z is at least 3,
t is an integer from 2 to 8,
Z⁵ is a radical of the formula VIII

—(OCH₂CH₂)ᵢ(OCH₂CH(CH₃))ⱼ—R^{q} (VIII)
in which
the order of the alkylene oxide units is arbitrary and i and j, independently of one another, are an integer from 0 to 200, where the sum of i and j is >O,
R^{q} is hydrogen or a C₁-C₈-alkyl radical
   and mixtures thereof.

According to a suitable embodiment, the polysiloxanes δ) of the general formula VI.1 have no alkylene oxide radicals of the general formula VII. These polysiloxanes c4) then preferably have a number-average molecular weight in the range from about 300 to 5000, preferably 400 to 3000.

Suitable polysiloxanes δ) which have no alkylene oxide radicals are, for example, the Tegomer® grades from Goldschmidt.

According to a further suitable embodiment, the polysiloxanes δ) are silicone poly(alkylene oxide) copolymers of the formula VI.1, where at least one or two radicals Z¹ and/or Z² are a radical of the general formula VII.

Preferably, in the formula VII, the sum of v and w is chosen so that the molecular weight of the polysiloxanes δ) is then in a range from about 300 to 30 000.

Preferably, the total number of alkylene oxide units in the polysiloxanes δ), i.e. the sum of v and w in the formula VII, is then in a range from about 3 to 200, preferably 5 to 180.

According to a further suitable embodiment, the polysiloxanes δ) are silicone poly(alkylene oxide) copolymers of the formula VI.2 which have at least one radical Z³ of the general formula VII.

Preferably, in the formula VII, the sum of v and w is then in turn chosen so that the molecular weight of the polysiloxanes δ) is then in a range from about 300 to 30 000. The total number of alkylene oxide units in the polysiloxanes δ), i.e. the sum of v and w in the formula VII, is then preferably likewise in a range from about 3 to 200, preferably 5 to 180.

Suitable silicone poly(alkylene oxide) copolymers δ), which are known under the international generic name dimethicone, are the Tegopren® grades from Goldschmidt, Belsil® 6031 from Wacker and Silvet® L from Witco.

According to a preferred embodiment, the polysiloxanes δ) are silicone poly(alkylene oxide) copolymers of the formula VI.2 which have at least one radical Z³, in which Z³ is NHR^{k} and R^{k} is hydrogen or a radical of the formula —(CH₂)ᵤ—NH₂. u is an integer from 1 to 10, preferably 2 to 6. These include, for example, the MAN and MAR grades from Hüls, and the Finish grades from Wacker, e.g. Finish WT 1270.

Preferably, the polysiloxanes δ) comprise at least one compound of the general formula VI.3. Preferably, in the formula VI.3, R^{l} and R^{m}, independently of one another, are a C₂-C₄-alkylene radical. In particular, R^{l} and R^{m}, independently of one another, are a C₂-C₃-alkylene radical.

Preferably, the molecular weight of the compound of the formula VI.3 is in a range from about 300 to 100 000.

Preferably, in the formula VI.3, p is an integer from 1 to 20, such as, for example, 2 to 10.

Preferably, the total number of alkylene oxide units in the compound of the formula VI.3, i.e. the sum of r and s, is in a range from about 3 to 200, preferably 5 to 180.

Preferably, the end groups of the polysiloxanes with repeat units of the general formula VI.3 are chosen from (CH₃)₃SiO, H, C₁-C₈-alkyl and mixtures thereof.

Compounds which contain amino groups and have repeat units of the general formula VI.3 preferably have an amine number in a range from about 2 to 50, in particular 3 to 20.

Suitable alkoxylated siloxane-amines of the formula VI.3 are described, for example, in WO-A-97/32917, which is hereby incorporated in its entirety by reference. Commercially available compounds are, for example, the Silsoft® grades from Witco, e.g. Silsoft® A-843.

Preferably, in the formula VI.4, the radical Rⁿ is a C₂-C₄-alkylene radical.

Preferably, in the formula VI.4 R^{o} and R^{p}, independently of one another, are hydrogen or C₁-C₄-alkyl.

Preferably, the sum of x, y and z is chosen so that the molecular weight of the compound of the formula VI.4 is in a range from about 300 to 100 000, preferably 500 to 50 000.

Preferably, the total number of the alkylene oxide units in the radical of the formula VIII, i.e. the sum of i and j, is in a range from about 3 to 200, preferably 5 to 80.

Preferably, in the formula VIII, the radical R^{q} is hydrogen or C₁-C₄-alkyl.

A suitable compound of the formula VI.4 is, for example, Silsoft® A-858 from Witco.

Suitable polysiloxanes δ) are also the polydimethylsiloxanes described in EP-A-277 816.

If appropriate, the urethane(meth)acrylates according to the invention additionally comprise at least one incorporated component which is chosen from
ε) compounds which comprise two or more active hydrogen atoms and at least one ionogenic and/or ionic group per molecule,
ζ) monohydric alcohols, amines with a primary or secondary amino group, aliphatic, cycloaliphatic or aromatic monoisocyanates and mixtures thereof,
η) α,β-ethylenically unsaturated compounds which additionally comprise at least one isocyanate group per molecule,
   and mixtures thereof.

Monomer d)

In a preferred embodiment, the copolymers according to the invention comprise, in copolymerized form, in addition to the abovementioned monomers a) to c), at least one further monomer d) which contains amide groups and is of the general formula I

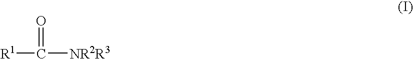

where

one of the radicals R¹ to R³ is a group of the formula CH₂═CR⁴— where R⁴═H or C₁-C₄-alkyl and the other radicals R¹ to R³, independently of one another, are H, alkyl, cycloalkyl, heterocycloalkyl, aryl or hetaryl,

where R¹ and R², together with the amide group to which they are bonded, may also be a lactam having 5 to 8 ring atoms,

where R² and R³, together with the nitrogen atom to which they are bonded, may also be a five- to seven-membered heterocycle,

with the proviso that the sum of the carbon atoms of the radicals R¹, R² and R³ is at most 8.

Preferably, in addition to the carbonyl carbon atom of the amide group, the compounds of component d) have at most 7 further carbon atoms.

Preferably, the compounds of component d) are chosen from primary amides of α,β-ethylenically unsaturated monocarboxylic acids, N-vinylamides of saturated monocarboxylic acids, N-vinyllactams, N-alkylamides and N,N-dialkylamides of α,β-ethylenically unsaturated monocarboxylic acids and mixtures thereof.

Preferred monomers d) are N-vinyllactams and derivatives thereof, which can, for example, have one or more C₁-C₆-alkyl substituents, such as methyl, ethyl, n-propyl, isopropyl, n-butyl, sec-butyl, tert-butyl etc. These include, for example, N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc.

Particular preference is given to using N-vinylpyrrolidone and N-vinylcaprolactam.

Suitable monomers d) are also acrylamide and methacrylamide.

Suitable N-alkylamides and N,N-dialkylamides of α,β-ethylenically unsaturated monocarboxylic acids which, in addition to the carbonyl carbon atom of the amide group, have at most 7 further carbon atoms are, for example, N-methyl(meth)acrylamide, N-ethyl(meth)acrylamide, N-propyl(meth)acrylamide, N-(n-butyl)(meth)acrylamide, N-tert-butyl(meth)acrylamide, n-pentyl(meth)acrylamide, n-hexyl(meth)acrylamide, n-heptyl(meth)acrylamide, N,N-dimethyl(meth)acrylamide, N,N-diethyl(meth)acrylamide, piperidinyl(meth)acrylamide, morpholinyl(meth)acrylamide and mixtures thereof.

Open-chain N-vinylamide compounds suitable as monomers d) are, for example, N-vinylformamide, N-vinyl-N-methylformamide, N-vinylacetamide, N-vinyl-N-methylacetamide, N-vinyl-N-ethylacetamide, N-vinylpropionamide, N-vinyl-N-methylpropionamide, N-vinylbutyramide and mixtures thereof. Preference is given to using N-vinylformamide.

Suitable monomers d) are also compounds of the formula

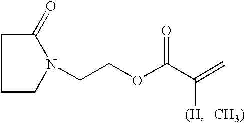

Particular preference is given to using N-vinylpyrrolidone, N-vinylcaprolactam, N-vinylformamide and the compounds of the above formula.

The copolymers used according to the invention comprise preferably 5 to 95% by weight, particularly preferably 10 to 90% by weight, based on the total weight of the compounds used for the polymerization, of at least one monomer d) in copolymerized form.

A specific embodiment relates to the use of copolymers which comprise no monomer d) in copolymerized form. These include, in particular, copolymers which comprise no N-vinylpyrrolidone and/or no N-vinylcaprolactam in copolymerized form.

Monomer e)

The copolymers A) according to the invention can additionally comprise at least one hydrophobic monomer e) in copolymerized form.

Preferably, copolymers which are free from silicone groups comprise 0.2 to 50% by weight, particularly preferably 0.5 to 40% by weight, in particular 1 to 30% by weight, based on the total weight of the compounds used for the polymerization, of at least one hydrophobic monomer e) (and/or at least one hydrophobic monomer f) and/or g) in copolymerized form). Preferably, the copolymers containing silicone groups comprise 0.1 to 30% by weight, particularly preferably 0.2 to 20% by weight, in particular 0.5 to 15% by weight, based on the total weight of the compounds used for the polymerization, of at least one hydrophobic monomer e) in copolymerized form.

Suitable compounds e) are chosen from compounds of the general formulae III a), III b), III c), III d) and III e)

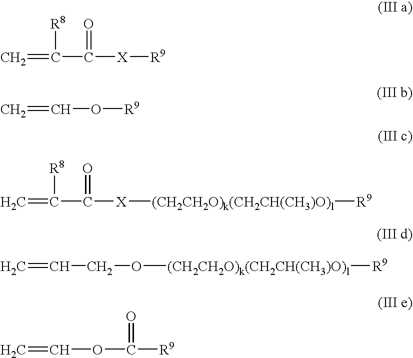

in which
the order of the alkylene oxide units is arbitrary,
k and l, independently of one another, are an integer from 0 to 1000, where the sum of k and l is at least 5,
R⁸ is hydrogen or C₁-C₄-alkyl, preferably methyl,
R⁹ is C₈-C₃₀-alkyl or C₈-C₃₀-alkenyl, and
X is O or a group of the formula NR¹⁰, in which R¹⁰ is H, alkyl, alkenyl, cycloalkyl, heterocycloalkyl, aryl or hetaryl.

If the copolymers used according to the invention are copolymers which are free from silicone groups, the compounds e) are specifically chosen from the compounds of the general formulae III a), III b) and III c).

Suitable monomers of the formula III a) in which X is O are, for example, n-octyl(meth)acrylate, 1,1,3,3-tetramethylbutyl(meth)acrylate, ethylhexyl(meth)acrylate, n-nonyl(meth)acrylate, n-decyl(meth)acrylate, n-undecyl(meth)acrylate, tridecyl(meth)acrylate, myristyl(meth)acrylate, pentadecyl(meth)acrylate, palmityl(meth)acrylate, heptadecyl(meth)acrylate, nonadecyl(meth)acrylate, arrachinyl(meth)acrylate, behenyl(meth)acrylate, lignocerenyl(meth)acrylate, cerotinyl(meth)acrylate, melissinyl(meth)acrylate, palmitoleinyl(meth)acrylate, oleyl(meth)acrylate, linolyl(meth)acrylate, linolenyl(meth)acrylate, stearyl(meth)acrylate, lauryl(meth)acrylate and mixtures thereof.

Suitable monomers of the formula III a) in which X is NR¹⁰ are, for example, n-octyl(meth)acrylamide, 1,1,3,3-tetramethylbutyl(meth)acrylamide, ethylhexyl(meth)acrylamide, n-nonyl(meth)acrylamide, n-decyl(meth)acrylamide, n-undecyl(meth)acrylamide, tridecyl(meth)acrylamide, myristyl(meth)acrylamide, pentadecyl(meth)acrylamide, palmityl(meth)acrylamide, heptadecyl(meth)acrylamide, nonadecyl(meth)acrylamide, arrachinyl(meth)acrylamide, behenyl(meth)acrylamide, lignocerenyl(meth)acrylamide, cerotinyl(meth)acrylamide, melissinyl(meth)acrylamide, palmitoleinyl(meth)acrylamide, oleyl(meth)acrylamide, linolyl(meth)acrylamide, linolenyl(meth)acrylamide, stearyl(meth)acrylamide, lauryl(meth)acrylamide, N-methyl-N-(n-octyl)(meth)acrylamide, N,N-di-(n-octyl)(meth)acrylamide and mixtures thereof.

Suitable monomers of the formula III b) are C₈-C₂₂-alkyl vinyl ethers, for example, n-octyl vinyl ether, 1,1,3,3-tetramethylbutyl vinyl ether, ethylhexyl vinyl ether, n-nonyl vinyl ether, n-decyl vinyl ether, n-undecyl vinyl ether, tridecyl vinyl ether, myristyl vinyl ether, pentadecyl vinyl ether, palmityl vinyl ether, heptadecyl vinyl ether, octadecyl vinyl ether, nonadecyl vinyl ether, arrachinyl vinyl ether, behenyl vinyl ether, lignocerenyl vinyl ether, cerotinyl vinyl ether, melissinyl vinyl ether, palmitoleinyl vinyl ether, oleyl vinyl ether, linolyl vinyl ether, linolenyl vinyl ether, stearyl vinyl ether, lauryl vinyl ether and mixtures thereof.

In the formulae III c) and III d), k is preferably an integer from 1 to 500, in particular 3 to 250. l is preferably an integer from 0 to 100.

R⁸ in the formula III c) is preferably hydrogen, methyl, ethyl, n-propyl, isopropyl, n-butyl, sec-butyl, tert-butyl, n-pentyl or n-hexyl, in particular hydrogen, methyl or ethyl.

R⁹ in the formulae III c) and III d) is preferably n-octyl, 1,1,3,3-tetramethylbutyl, ethylhexyl, n-nonyl, n-decyl, n-undecyl, tridecyl, myristyl, pentadecyl, palmityl, heptadecyl, octadecyl, nonadecyl, arrachinyl, behenyl, lignocerenyl, cerotinyl, melissinyl, palmitoleinyl, oleyl, linolyl, linolenyl, stearyl, lauryl.

Preferably, X in the formula III c) is O or NH.

Suitable polyether acrylates III c) are, for example, the polycondensation products of the abovementioned α,β-ethylenically unsaturated mono- and/or dicarboxylic acids and the acid chlorides, acid amides and anhydrides with polyetherols thereof. Suitable polyetherols can be prepared easily by reacting ethylene oxide, 1,2-propylene oxide and/or epichlorohydrin with a starter alcohol R⁹—OH. The alkylene oxides can be used individually, alternately one after the other or as a mixture. The polyether acrylates III c) can be used on their own or in mixtures for the preparation of the polymers used according to the invention.

Suitable allyl alcohol alkoxylates III d) are, for example, the etherification products of allyl chloride with corresponding polyetherols. Suitable polyetherols can be prepared easily by reacting ethylene oxide, 1,2-propylene oxide and/or epichlorohydrin with a starter alcohol R⁹—OH. The alkylene oxides can be used individually, alternately one after the other or as a mixture. The allyl alcohol alkoxylates III d) can be used on their own or in mixtures for the preparation of the polymers used according to the invention.

Suitable monomers III e) are C₈-C₃₀-, preferably C₈-C₂₂-carboxylic acid vinyl esters. These include, for example, n-octyl vinyl ester, 1,1,3,3-tetramethylbutyl vinyl ester, ethylhexyl vinyl ester, n-nonyl vinyl ester, n-decyl vinyl ester, n-undecyl vinyl ester, tridecyl vinyl ester, myristyl vinyl ester, pentadecyl vinyl ester, palmityl vinyl ester, heptadecyl vinyl ester, octadecyl vinyl ester, nonadecyl vinyl ester, arrachinyl vinyl ester, behenyl vinyl ester, lignocerenyl vinyl ester, cerotinyl vinyl ester, melissinyl vinyl ester, palmitoleinyl vinyl ester, oleyl vinyl ester, linolyl vinyl ester, linolenyl vinyl ester, stearyl vinyl ester, lauryl vinyl ester and mixtures thereof.

Monomer f)

The copolymers used according to the invention can additionally comprise, in copolymerized form, at least one monomer f) which is chosen from esters of α,β-ethylenically unsaturated mono- and dicarboxylic acids with C₁-C₇-alkanols which are different from component e), polyether acrylates which are different from III c), C₁-C₇-alkyl vinyl ethers and allyl alcohol alkoxylates which are different from III d) and esters of vinyl alcohol with C₁-C₇-monocarboxylic acids.

The fraction of monomers f) (and/or g) is preferably up to 40% by weight, based on the total weight of the compounds used for the polymerization. A suitable use amount of additional monomers f) is in a range from 0.1 to 30% by weight, in particular 1 to 25% by weight, based on the total weight of the compounds used for the polymerization.

Preferably, the compound f) is chosen from compounds of the general formulae III a*), III b*), III c*), III d*) and III e*)

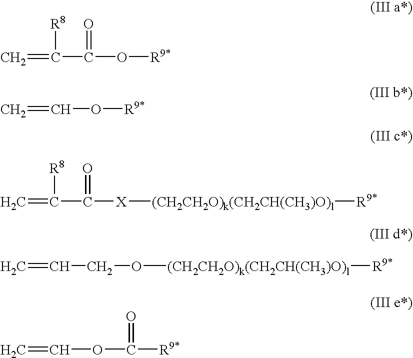

in which
the order of the alkylene oxide units is arbitrary,
k and l, independently of one another, are an integer from 0 to 1000, where the sum of k and l is at least 5,
R⁸ is hydrogen or C₁-C₄-alkyl, preferably methyl,
R⁹* is hydrogen, C₁-C₈-alkyl or C₃-C₈-alkenyl, and
X is O or a group of the formula NR¹⁰, in which R¹⁰ is H, alkyl, alkenyl, cycloalkyl, heterocycloalkyl, aryl or hetaryl.

Suitable esters of α,β-ethylenically unsaturated mono- and dicarboxylic acids with C₁-C₇-alkanols are, for example, methyl(meth)acrylate, methyl ethacrylate, ethyl(meth)acrylate, ethyl ethacrylate, n-butyl(meth)acrylate, tert-butyl(meth)acrylate, tert-butyl ethacrylate, n-pentyl(meth)acrylate, n-hexyl(meth)acrylate, n-heptyl(meth)acrylate, etc. Preferred monomers f) are the esters of α,β-ethylenically unsaturated mono- and dicarboxylic acids with C₁-C₃-alkanols, in particular methyl methacrylate.

In the formulae III c*) and III d*), k is preferably an integer from 1 to 500, in particular 3 to 250. Preferably, l is an integer from 0 to 100.

Preferably, R⁸ in the formula III c*) is hydrogen, methyl, ethyl, n-propyl, isopropyl, n-butyl, sec-butyl, tert-butyl, n-pentyl or n-hexyl, in particular hydrogen, methyl or ethyl.

Preferably, R⁹* in the formulae III c*) and III d*) is hydrogen, methyl, ethyl, n-propyl, isopropyl, n-butyl, sec-butyl, tert-butyl, n-pentyl or n-hexyl, in particular hydrogen, methyl or ethyl.

Preferably, X in the formula III c*) is O or NH.

Suitable polyether acrylates III c*) are, for example, the polycondensation products of the abovementioned α,β-ethylenically unsaturated mono- and/or dicarboxylic acids and the acid chlorides, acid amides and anhydrides with polyetherols thereof. Suitable polyetherols can be prepared easily by reacting ethylene oxide, 1,2-propylene oxide and/or epichlorohydrin with water or a starter alcohol R⁹*-OH. The alkylene oxides can be used individually, alternately one after the other or as a mixture. The polyether acrylates III c*) can be used on their own or in mixtures for the preparation of the polymers used according to the invention.

Suitable allyl alcohol alkoxylates III d*) are, for example, the etherification products of allyl chloride with corresponding polyetherols. Suitable polyetherols can be prepared easily by reacting ethylene oxide, 1,2-propylene oxide and/or epichlorohydrin with water or a starter alcohol R⁹*-OH. The alkylene oxides can be used individually, alternately one after the other or as a mixture. The allyl alcohol alkoxylates III d*) can be used on their own or in mixtures for the preparation of the polymers used according to the invention.

Suitable esters of vinyl alcohol with C₁-C₇-monocarboxylic acids III e*) are, for example, vinyl acetate, vinyl propionate, vinyl butyrate and mixtures thereof.

Monomer g)

The copolymers A) used according to the invention can additionally comprise, in copolymerized form, at least one monomer g) which is different from the components a) to f) and is copolymerizable therewith.

Preferably, the fraction of monomers g (and/or f) is up to 40% by weight, based on the total weight of the compounds used for the polymerization. A suitable use amount for additional monomers g) is in a range from 0.1 to 25% by weight, in particular 0.5 to 20% by weight, based on the total weight of the compounds used for the polymerization.

Preferably, the component f) is chosen from esters of α,β-ethylenically unsaturated mono- and dicarboxylic acids with C₂-C₃₀-diols, amides of α,β-ethylenically unsaturated mono- and dicarboxylic acids with C₂-C₃₀-amino alcohols which have a primary or secondary amino group, esters of allyl alcohol with C₁-C₇-monocarboxylic acids, polyether acrylates different from III c) and III c*), vinylaromatics, vinyl halides, vinylidene halides, C₂-C₈-monoolefins, nonaromatic hydrocarbons having at least two conjugated double bonds and mixtures thereof.

Suitable additional monomers g) are also 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate and 3-hydroxy-2-ethylhexyl methacrylate.

Suitable additional monomers g) are also 2-hydroxyethylacrylamide, 2-hydroxyethylmethacrylamide, 2-hydroxyethylethacrylamide, 2-hydroxypropylacrylamide, 2-hydroxypropylmethacrylamide, 3-hydroxypropylacrylamide, 3-hydroxypropylmethacrylamide, 3-hydroxybutylacrylamide, 3-hydroxybutylmethacrylamide, 4-hydroxybutylacrylamide, 4-hydroxybutylmethacrylamide, 6-hydroxyhexylacrylamide, 6-hydroxyhexylmethacrylamide, 3-hydroxy-2-ethylhexylacrylamide and 3-hydroxy-2-ethylhexylmethacrylamide.

Suitable polyether acrylates g) are also urethane(meth)acrylates with alkylene oxide groups. Such compounds are described in DE 198 38 851 (component e2)), which is hereby incorporated in its entirety by reference.

Suitable additional monomers g) are also ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

The abovementioned additional monomers g) can in each case be used individually or in the form of any desired mixtures.

Preference is given to the use of an ampholytic copolymer which is obtainable by free-radical copolymerization of
1 to 99% by weight, preferably 2 to 96% by weight, of at least one compound a1) with a free-radically polymerizable, α,β-ethylenically unsaturated double bond and at least one anionogenic and/or anionic group per molecule, preferably acrylic acid and/or methacrylic acid,
2 to 96% by weight of at least one compound a2), preferably chosen from N-vinylimidazole compounds, N-[3-(dimethylamino)propyl]acrylamide, N-[3-(dimethylamino)propyl]methacrylamide, N,N-dimethylaminoethyl acrylate, N,N-dimethylaminoethyl methacrylate and mixtures thereof,
0.05 to 5% by weight of at least one crosslinker b), preferably ethylene glycol di(meth)acrylate and/or pentaerythritol triallyl ether,
0 to 30% by weight, preferably 0 to 15% by weight, of at least one silicone compound c),
0 to 95% by weight of at least one amide-group-containing monomer d), preferably vinylpyrrolidone and/or vinylcaprolactam,
0 to 40% by weight of at least one compound e) which is preferably chosen from C₈-C₂₂-(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,
0 to 40% by weight of at least one monomer f) which is preferably chosen from C₁-C₆-(meth)acrylates, in particular methyl(meth)acrylate, ethyl(meth)acrylate, n-butyl(meth)acrylate and mixtures thereof.

A specific embodiment is the use of ampholytic copolymers which comprise no amide-group-containing monomer d) in copolymerized form. These specifically include copolymers which comprise no vinylpyrrolidone and/or no vinylcaprolactam in copolymerized form. A very specific embodiment is the use according to the invention of vinylpyrrolidone-free copolymers.

Preference is given to the use of a copolymer which comprises no monomer d) (specifically no vinylpyrrolidone and/or vinylcaprolactam) in copolymerized form which comprises
1 to 99% by weight, preferably 2 to 96% by weight, of at least one compound a1) with a free-radically polymerizable, α,β-ethylenically unsaturated double bond and at least one anionogenic and/or anionic group per molecule, preferably acrylic acid and/or methacrylic acid,
1 to 30% by weight of at least one compound a2), preferably chosen from N-vinylimidazole compounds, N-[3-(dimethylamino)propyl]acrylamide, N-[3-(dimethylamino)propyl]methacrylamide, N,N-dimethylaminoethyl acrylate, N,N-dimethylaminoethyl methacrylate and mixtures thereof,
0.1 to 2% by weight of at least one crosslinker b), preferably ethylene glycol di(meth)acrylate and/or pentaerythritol triallyl ether,
0 to 3% by weight, preferably 0 to 15% by weight, of at least one silicone compound c),
   in copolymerized form.

The abovementioned copolymers can additionally comprise
up to 40% by weight of at least one compound e) which is preferably chosen from C₈-C₂₂-(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof, and/or
up to 40% by weight of at least one monomer f) which is preferably chosen from C₁-C₆-(meth)acrylates, in particular methyl(meth)acrylate, ethyl(meth)acrylate, n-butyl(meth)acrylate and mixtures thereof,
   in copolymerized form.

Preference is given to the use of an ampholytic copolymer which is obtainable by free-radical copolymerization of
a1) at least one compound with a free-radically polymerizable, α,β-ethylenically unsaturated double bond and at least one anionogenic and/or anionic group per molecule,
a2) at least one N-vinylimidazole compound,
b) at least one free-radically polymerizable crosslinking compound which comprises at least two α,β-ethylenically unsaturated double bonds per molecule,
   where at least some of the compounds a1) and a2) are used in the form of a monomer pair where the molar ratio of anionogenic groups of component a1) to cationogenic groups of component a2) is about 1:1. Specifically, these are copolymers described in the German patent application 10 2005 034 412.7.

A specific embodiment is the use of anionically ampholytic copolymers. These comprise, as component a1), preferably acrylic acid, methacrylic acid or a mixture thereof. The component a2) is preferably chosen from N-vinylimidazole, N-[3-(dimethylamino)propyl]acrylamide, N-[3-(dimethylamino)propyl]methacrylamide, N,N-dimethylaminoethyl acrylate, N,N-dimethylaminoethyl methacrylate and mixtures thereof. Preference is given to the use of copolymers which have a molar excess of anionogenic/anionic groups compared with cationogenic/cationic groups of more than 1:1, particularly preferably at least 1.2:1, in particular at least 1.4:1.

Preference is given to the use of an ampholytic copolymer which is obtainable by free-radical copolymerization of
methacrylic acid and/or acrylic acid a1),
at least one compound a2) chosen from N-vinylimidazole, N-[3-(dimethylamino)propyl]acrylamide, N-[3-(dimethylamino)propyl]methacrylamide and mixtures thereof,
at least one crosslinker b), preferably ethylene glycol di(meth)acrylate and/or pentaerythritol triallyl ether,
if appropriate at least one silicone compound c),
   where the molar ratio of anionogenic/anionic groups to cationogenic/cationic groups is at least 1:1.

Preference is given to the use of an ampholytic copolymer which is obtainable by free-radical copolymerization of
methacrylic acid and/or acrylic acid a1),
at least one compound a2) chosen from N-vinylimidazole, N-[3-(dimethylamino)propyl]acrylamide, N-[3-(dimethylamino)propyl]methacrylamide and mixtures thereof,
at least one crosslinker b), preferably ethylene glycol di(meth)acrylate and/or pentaerythritol triallyl ether,
if appropriate at least one silicone compound c),
3 to 35% by weight, based on the total weight of the monomers used for the polymerization, of at least one monomer f) which is preferably chosen from C₁-C₆-(meth)acrylates,
   where the molar ratio of anionogenic/anionic groups to cationogenic/cationic groups is at least 1.2:1.

Preference is given to the use of an ampholytic copolymer which is obtainable by free-radical copolymerization of
methacrylic acid and/or acrylic acid a1),
at least one compound a2) chosen from N-vinylimidazole, N-[3-(dimethylamino)propyl]acrylamide, N-[3-(dimethylamino)propyl]methacrylamide and mixtures thereof,
at least one crosslinker b), preferably ethylene glycol di(meth)acrylate and/or pentaerythritol triallyl ether,
if appropriate at least one silicone compound c),
0.1 to 10% by weight, based on the total weight of the monomers used for the polymerization, of at least one compound e) which is preferably chosen from C₈-C₂₂-(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,
where the molar ratio of anionogenic/anionic groups to cationogenic/cationic groups is at least 1.4:1.
   In the case of the three last-mentioned anionically ampholytic copolymers, up to 60% by weight of component a1), based on the total weight of the monomers a1), can be replaced by at least one monomer d), preferably vinylpyrrolidone and/or vinylcaprolactam.

Preference is also given to anionically ampholytic copolymers A) for whose preparation at least part of the monomers a1) and a2) is used in the form of a monomer pair.

A specific embodiment is the use of silicone-group-free anionically ampholytic copolymers.

Particular preference is given to the use of an ampholytic copolymer which is obtainable by free-radical copolymerization of
at least 2% by weight, based on the total weight of the monomers used for the polymerization, of at least one monomer pair from N-vinylimidazole and acrylic acid and/or methacrylic acid,
5 to 70% by weight of methacrylic acid and/or acrylic acid,
0.1 to 2% by weight of at least one crosslinker b), preferably ethylene glycol di(meth)acrylate and/or pentaerythritol triallyl ether,
20 to 95% by weight of vinylpyrrolidone and/or vinylcaprolactam,
0 to 40% by weight of at least one further monomer which is chosen from methyl(meth)acrylate, ethyl(meth)acrylate, n-butyl(meth)acrylate, C₈-C₂₂-(meth)acrylates, polyether(meth)acrylates terminated with C₈-C₂₂-alkyl groups, C₈-C₂₂-carboxylic acid vinyl esters and mixtures thereof.

N-[3-(dimethylamino)propyl]acrylamide and/or N-[3-(dimethylamino)propyl]methacrylamide can be used instead of or in addition to vinylimidazole.

Particular preference is also given to the use of an ampholytic copolymer which is obtainable by free-radical copolymerization of
at least 2% by weight, based on the total weight of the monomers used for the polymerization, of at least one monomer pair from N-vinylimidazole and acrylic acid and/or methacrylic acid,
5 to 70% by weight of methacrylic acid and/or acrylic acid,
0.1 to 2% by weight of at least one crosslinker b), preferably ethylene glycol di(meth)acrylate and/or pentaerythritol triallyl ether,
20 to 85% by weight of vinylpyrrolidone and/or vinylcaprolactam.

N-[3-(dimethylamino)propyl]acrylamide and/or N-[3-(dimethylamino)propyl]methacrylamide can be used instead of or in addition to vinylimidazole.

Particular preference is given to the use of an ampholytic copolymer which is obtainable by free-radical copolymerization of
at least 5% by weight, based on the total weight of the monomers used for the polymerization, of at least one monomer pair from N-vinylimidazole and acrylic acid and/or methacrylic acid,
5 to 70% by weight of methacrylic acid and /or acrylic acid,
0.1 to 2% by weight of at least one crosslinker b), preferably ethylene glycol di(meth)acrylate and/or pentaerythritol triallyl ether,
20 to 85% by weight of vinylpyrrolidone and/or vinylcaprolactam,
5 to 40% by weight of at least one further monomer which is chosen from C₁-C₆-(meth)acrylates, in particular methyl(meth)acrylate, ethyl(meth)acrylate, n-butyl(meth)acrylate and mixtures thereof.

N-[3-(dimethylamino)propyl]acrylamide and/or N-[3-(dimethylamino)propyl]methacrylamide can be used instead of or in addition to vinylimidazole.

Particular preference is given to the use of an ampholytic copolymer which is obtainable by free-radical copolymerization of
at least 5% by weight, based on the total weight of the monomers used for the polymerization, of at least one monomer pair from N-vinylimidazole and acrylic acid and/or methacrylic acid,
5 to 70% by weight of methacrylic acid and/or acrylic acid,
0.1 to 2% by weight of at least one crosslinker b), preferably ethylene glycol di(meth)acrylate and/or pentaerythritol triallyl ether,
20 to 85% by weight of vinylpyrrolidone and/or vinylcaprolactam,
0.5 to 20% by weight, preferably 1 to 10% by weight, of at least one further monomer which is chosen from C₈-C₃₀-(meth)acrylates, polyether(meth)acrylates terminated with C₈-C₃₀-alkyl groups, and mixtures thereof, in particular with stearyl methacrylate, polyethylene glycol(meth)acrylates terminated with C₁₈-C₂₂-alkyl groups, and mixtures thereof.

N-[3-(dimethylamino)propyl]acrylamide and/or N-[3-(dimethylamino)propyl]methacrylamide can be used instead of or in addition to vinylimidazole.

In a specific embodiment, all of the abovementioned anionic, ampholytic copolymers which are free from silicone groups and which comprise at least 5% by weight of at least one vinylimidazole compound in copolymerized form are subjected to partial or complete quaternization. Suitable quaternizing agents are specified below.

Preference is also given to the use of an anionic, ampholytic copolymer containing silicone groups which is obtainable by free-radical copolymerization of
a1) at least one compound with a free-radically polymerizable, α,β-ethylenically unsaturated double bond and at least one anionogenic and/or anionic group per molecule,
a2) at least one compound with a free-radically polymerizable, α,β-ethylenically unsaturated double bond and at least one cationogenic and/or cationic group per molecule,
b) at least one free-radically polymerizable crosslinking compound which comprises at least two α,β-ethylenically unsaturated double bonds per molecule,
in the presence of at least one silicone compound c) comprising a polyether group and/or a free-radically polymerizable olefinically unsaturated double bond. Specifically, these are copolymers described in the German patent application 10 2005 034 412.7.

Particular preference is given to the use of an ampholytic copolymer containing silicone groups which is obtainable by free-radical copolymerization of
at least 2% by weight, based on the total weight of the monomers used for the polymerization, of at least one monomer pair from N-vinylimidazole a2) and acrylic acid and/or methacrylic acid a1),
5 to 70% by weight of methacrylic acid and/or acrylic acid a1),
0.1 to 2% by weight of at least one crosslinker b), preferably ethylene glycol di(meth)acrylate and/or pentaerythritol triallyl ether,
0.05 to 30% by weight, particularly preferably 0.1 to 20% by weight, in particular 0.1 to 15% by weight, of at least one silicone compound c),
20 to 95% by weight of vinylpyrrolidone and/or vinylcaprolactam d),
0.1 to 20% by weight of at least one compound e) which is preferably chosen from C₈-C₂₂-(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,
5 to 40% by weight of at least one monomer f) which is preferably chosen from C₁-C₆-(meth)acrylates, in particular methyl(meth)acrylate, ethyl(meth)acrylate, n-butyl(meth)acrylate and mixtures thereof.

N-[3-(dimethylamino)propyl]acrylamide and/or N-[3-(dimethylamino)propyl]methacrylamide can be used instead of or in addition to vinylimidazole.

Particular preference is also given to the use of an ampholytic copolymer containing silicone groups which is obtainable by free-radical copolymerization of
at least 5% by weight, based on the total weight of the monomers used for the polymerization, of at least one monomer pair from N-vinylimidazole a2) and acrylic acid and/or methacrylic acid a1),
5 to 70% by weight of methacrylic acid and/or acrylic acid a1),
0.1 to 2% by weight of at least one crosslinker b), preferably ethylene glycol di(meth)acrylate and/or pentaerythritol triallyl ether,
0.05 to 30% by weight, particularly preferably 0.1 to 20% by weight, in particular 0.1 to 15% by weight, of at least one silicone compound c),
20 to 85% by weight of vinylpyrrolidone and/or vinylcaprolactam d).

N-[3-(dimethylamino)propyl]acrylamide and/or N-[3-(dimethylamino)propyl]methacrylamide can be used instead of or in addition to vinylimidazole.

Particular preference is also given to the use of an ampholytic copolymer containing silicone groups which is obtainable by free-radical copolymerization of
at least 5% by weight, based on the total weight of the monomers used for the polymerization, of at least one monomer pair from N-vinylimidazole a2) and acrylic acid and/or methacrylic acid a1),
5 to 70% by weight of methacrylic acid and/or acrylic acid a1),
0.1 to 2% by weight of at least one crosslinker b), preferably ethylene glycol di(meth)acrylate and/or pentaerythritol triallyl ether,
0.05 to 30% by weight, particularly preferably 0.1 to 20% by weight, in particular 0.1 to 15% by weight, of at least one silicone compound c),
20 to 85% by weight of vinylpyrrolidone and/or vinylcaprolactam d),
5 to 40% by weight of at least one further monomer f), which is preferably chosen from C₁-C₆-(meth)acrylates, in particular methyl(meth)acrylate, ethyl(meth)acrylate, n-butyl(meth)acrylate and mixtures thereof.

N-[3-(dimethylamino)propyl]acrylamide and/or N-[3-(dimethylamino)propyl]methacrylamide can be used instead of or in addition to vinylimidazole.

Particular preference is also given to the use of an ampholytic copolymer containing silicone groups which is obtainable by free-radical copolymerization of
at least 5% by weight, based on the total weight of the monomers used for the polymerization, of at least one monomer pair from N-vinylimidazole a2) and acrylic acid and/or methacrylic acid a1),
5 to 70% by weight of methacrylic acid and/or acrylic acid a1),
0.1 to 2% by weight of at least one crosslinker b), preferably ethylene glycol di(meth)acrylate and/or pentaerythritol triallyl ether,
0.05 to 30% by weight, particularly preferably 0.1 to 20% by weight, in particular 0.1 to 15% by weight, of at least one silicone compound c),
20 to 85% by weight of vinylpyrrolidone and/or vinylcaprolactam d),
0.5 to 20% by weight, preferably 1 to 10% by weight, of at least one further monomer which is chosen from C₈-C₂₂-(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, and mixtures thereof, in particular from stearyl methacrylate, polyethylene glycol(meth)acrylates terminated with C₁₈-C₂₂-alkyl groups, and mixtures thereof.

N-[3-(dimethylamino)propyl]acrylamide and/or N-[3-(dimethylamino)propyl]methacrylamide can be used instead of or in addition to vinylimidazole.

In a specific embodiment, all of the abovementioned anionic copolymers A) containing silicone groups which comprise at least 5% by weight of at least one vinylimidazole compound in copolymerized form are subjected to a partial or complete quaternization. Suitable quaternizing agents are those specified below.

A further specific embodiment is the use of cationically ampholytic copolymers. These comprise, as component a1), preferably acrylic acid, methacrylic acid or a mixture thereof. N-vinylimidazole and mixtures of N-vinylimidazole with N-[3-(dimethylamino)propyl]acrylamide and/or N-[3-(dimethylamino)propyl]methacrylamide are preferred as component a2).

Preferred cationically ampholytic copolymers A) are obtainable by free-radical copolymerization of
methacrylic acid and/or acrylic acid a1),
N-vinylimidazole a2),
at least one crosslinker b), preferably ethylene glycol di(meth)acrylate and/or pentaerythritol triallyl ether,
if appropriate at least one silicone compound c),
   where the molar ratio of cationogenic/cationic groups to anionogenic/anionic groups is at least 6:1.

Preferred cationically ampholytic copolymers A) are obtainable by free-radical copolymerization of
methacrylic acid and/or acrylic acid a1),
N-vinylimidazole a2),
at least one crosslinker b), preferably ethylene glycol di(meth)acrylate and/or pentaerythritol triallyl ether,
if appropriate at least one silicone compound c),
3 to 35% by weight, based on the total weight of the monomers used for the polymerization, of at least one monomer f) which is preferably chosen from C₁-C₆-(meth)acrylates,
   where the molar ratio of cationogenic/cationic groups to anionogenic/anionic groups is at least 6:1.

Preferred cationically ampholytic copolymers A) are obtainable by free-radical copolymerization of
methacrylic acid and/or acrylic acid a1),
N-vinylimidazole a2),
at least one crosslinker b), preferably ethylene glycol di(meth)acrylate and/or pentaerythritol triallyl ether,
if appropriate at least one silicone compound c),
0.1 to 10% by weight of at least one compound e) which is preferably chosen from C₈-C₂₂-(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,
   where the molar ratio of cationogenic/cationic groups to anionogenic/anionic groups is at least 6:1.

In the case of the three last-mentioned cationically ampholytic copolymers, up to 50% by weight of the N-vinylimidazole a2), based on the total weight of the component a2), can be replaced by another monomer, preferably N-[3-(dimethylamino)propyl]acrylamide and/or N-[3-(dimethylamino)propyl]methacrylamide.

Preference is also given to cationically ampholytic copolymers A) for whose preparation at least part of the monomers a1) and a2) are used in the form of a monomer pair.

A specific embodiment is the use of silicone-group-free cationically ampholytic copolymers.

Particular preference is given to the use of an ampholytic copolymer which is obtainable by free-radical copolymerization of
at least 2% by weight, based on the total weight of the monomers used for the polymerization, of at least one monomer pair of N-vinylimidazole and acrylic acid and/or methacrylic acid,
3 to 70% by weight of at least one monomer with a cationogenic or cationic group, preferably chosen from vinylimidazole compounds and mixtures of at least one vinylimidazole compound with at least one compound which is chosen from N-[3-(dimethylamino)propyl]acrylamide, N-[3-(dimethylamino)propyl]methacrylamide, N,N-dimethylaminoethyl(meth)acrylate, N,N-dimethylaminopropyl(meth)acrylate and mixtures thereof,
0.1 to 2% by weight of at least one crosslinker b), preferably ethylene glycol di(meth)acrylate and/or pentaerythritol triallyl ether,
0 to 95% by weight, preferably 20 to 95% by weight, of vinylpyrrolidone and/or vinylcaprolactam,
0 to 40% by weight of at least one further monomer which is chosen from methyl(meth)acrylate, ethyl(meth)acrylate, n-butyl(meth)acrylate, C₈-C₂₂-(meth)acrylates, polyether(meth)acrylates terminated with C₈-C₂₂-alkyl groups, C₈-C₂₂-carboxylic acid vinyl esters and mixtures thereof.

Particular preference is also given to the use of an ampholytic copolymer which is obtainable by free-radical copolymerization of
at least 2% by weight, based on the total weight of the monomers used for the polymerization, of at least one monomer pair of N-vinylimidazole and acrylic acid and/or methacrylic acid,
3 to 70% by weight of at least one monomer with a cationogenic or cationic group, preferably chosen from vinylimidazole compounds and mixtures of at least one vinylimidazole compound with at least one compound which is chosen from N-[3-(dimethylamino)propyl]acrylamide, N-[3-(dimethylamino)propyl]methacrylamide, N,N-dimethylaminoethyl(meth)acrylate, N,N-dimethylaminopropyl(meth)acrylate and mixtures thereof,
0.1 to 2% by weight of at least one crosslinker b), preferably ethylene glycol di(meth)acrylate and/or pentaerythritol triallyl ether,
20 to 85% by weight of vinylpyrrolidone and/or vinylcaprolactam.

Particular preference is given to the use of an ampholytic copolymer which is obtainable by free-radical copolymerization of
at least 2% by weight, based on the total weight of the monomers used for the polymerization, of at least one monomer pair of N-vinylimidazole and acrylic acid and/or methacrylic acid,

3 to 50% by weight of at least one monomer with a cationogenic or cationic group, preferably chosen from vinylimidazole compounds and mixtures of at least one vinylimidazole compound with at least one compound which is chosen from N-[3-(dimethylamino)propyl]acrylamide, N-[3-(dimethylamino)propyl]methacrylamide, N,N-dimethylaminoethyl(meth)acrylate, N,N-dimethylaminopropyl(meth)acrylate and mixtures thereof,
0.1 to 2% by weight of at least one crosslinker b), preferably ethylene glycol di(meth)acrylate and/or pentaerythritol triallyl ether,
20 to 95% by weight of vinylpyrrolidone and/or vinylcaprolactam,
5 to 40% by weight of at least one further monomer which is chosen from C₁-C₆-(meth)acrylates, in particular methyl(meth)acrylate, ethyl(meth)acrylate, n-butyl(meth)acrylate and mixtures thereof.

Particular preference is given to the use of an ampholytic copolymer which is obtainable by free-radical copolymerization of
at least 5% by weight, based on the total weight of the monomers used for the polymerization, of at least one monomer pair of N-vinylimidazole and acrylic acid and/or methacrylic acid,
3 to 70% by weight of at least one monomer with a cationogenic or cationic group, preferably chosen from vinylimidazole compounds and mixtures of at least one vinylimidazole compound with at least one compound which is chosen from N-[3-(dimethylamino)propyl]acrylamide, N-[3-(dimethylamino)propyl]methacrylamide, N,N-dimethylaminoethyl(meth)acrylate, N,N-dimethylaminopropyl(meth)acrylate and mixtures thereof,
0.1 to 2% by weight of at least one crosslinker b), preferably ethylene glycol di(meth)acrylate and/or pentaerythritol triallyl ether,
20 to 85% by weight of vinylpyrrolidone and/or vinylcaprolactam,
0.5 to 20% by weight, preferably 1 to 10% by weight, of at least one further monomer which is chosen from C₈-C₃₀-(meth)acrylates, polyether(meth)acrylates terminated with C₈-C₃₀-alkyl groups and mixtures thereof, in particular from stearyl methacrylate, polyethylene glycol(meth)acrylates terminated with C₁₈-C₂₂-alkyl groups and mixtures thereof.

In a specific embodiment, all of the abovementioned silicone-group-free cationic, ampholytic copolymers are subjected to partial or complete quaternization. Suitable quaternizing agents are those specified below.

In a specific embodiment, the abovementioned silicone-group-free cationic, ampholytic copolymers can comprise, in copolymerized form and instead of N-vinylimidazole, a component a2) which consists of N-[3-(dimethylamino)propyl]acrylamide and/or N-[3-(dimethylamino)propyl]methacrylamide or comprises N-[3-(dimethylamino)propyl]acrylamide and/or N-[3-(dimethylamino)propyl]methacrylamide.

Preference is also given to the use of a silicone-group-containing cationic, ampholytic copolymer which is obtainable by free-radical copolymerization of
a1) at least one compound with a free-radically polymerizable, α,β-ethylenically unsaturated double bond and at least one anionogenic and/or anionic group per molecule,
a2) at least one compound with a free-radically polymerizable, α,β-ethylenically unsaturated double bond and at least one cationogenic and/or cationic group per molecule,
b) at least one free-radically polymerizable crosslinking compound which comprises at least two α,β-ethylenically unsaturated double bonds per molecule,
   in the presence of at least one silicone compound c) comprising a polyether group and/or a free-radically polymerizable olefinically unsaturated double bond. Specifically, these are the copolymers described in the German patent application 10 2005 034 412.7.

Particular preference is given to the use of a silicone-group-containing ampholytic copolymer which is obtainable by free-radical copolymerization of
at least 2% by weight, based on the total weight of the monomers used for the polymerization, of at least one monomer pair of N-vinylimidazole a2) and acrylic acid and/or methacrylic acid a1),
3 to 70% by weight of at least one monomer with a cationogenic or cationic group, preferably chosen from vinylimidazole compounds and mixtures of at least one vinylimidazole compound with at least one compound which is chosen from N-[3-(dimethylamino)propyl]acrylamide, N-[3-(dimethylamino)propyl]methacrylamide, N,N-dimethylaminoethyl(meth)acrylate, N,N-dimethylaminopropyl(meth)acrylate and mixtures thereof,
0.1 to 2% by weight of at least one crosslinker b), preferably ethylene glycol di(meth)acrylate and/or pentaerythritol triallyl ether,
0.05 to 30% by weight, particularly preferably 0.1 to 20% by weight, in particular 0.1 to 15% by weight, of at least one silicone compound c),
0 to 95% by weight, preferably 20 to 95% by weight, of vinylpyrrolidone and/or vinylcaprolactam d),
0 to 20% by weight of at least one compound e) which is preferably chosen from C₈-C₂₂-(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,
0 to 40% by weight of at least one monomer f) which is preferably chosen from C₁-C₆-(meth)acrylates, in particular methyl(meth)acrylate, ethyl(meth)acrylate, n-butyl(meth)acrylate and mixtures thereof.

Particular preference is also given to the use of a silicone-group-containing ampholytic copolymer which is obtainable by free-radical copolymerization of
at least 2% by weight, based on the total weight of the monomers used for the polymerization, of at least one monomer pair of N-vinylimidazole a2) and acrylic acid and/or methacrylic acid a1),
3 to 70% by weight of at least one additional monomer a2) with a cationogenic or cationic group, preferably chosen from vinylimidazole compounds and mixtures of at least one vinylimidazole compound with at least one compound which is chosen from N-[3-(dimethylamino)propyl]acrylamide, N-[3-(dimethylamino)propyl]methacrylamide, N,N-dimethylaminoethyl(meth)acrylate, N,N-dimethylaminopropyl(meth)acrylate and mixtures thereof,
0.1 to 2% by weight of at least one crosslinker b), preferably ethylene glycol di(meth)acrylate and/or pentaerythritol triallyl ether,
0.05 to 30% by weight, particularly preferably 0.1 to 20% by weight, in particular 0.1 to 15% by weight, of at least one silicone compound c),
20 to 95% by weight of vinylpyrrolidone and/or vinylcaprolactam d).

Particular preference is also given to the use of an ampholytic copolymer containing silicone group which is obtainable by free-radical copolymerization of
at least 2% by weight, based on the total weight of the monomers used for the polymerization, of at least one monomer pair of N-vinylimidazole a2) and acrylic acid and/or methacrylic acid a1),
3 to 50% by weight of at least one additional monomer a2) with a cationogenic or cationic group, preferably chosen from vinylimidazole compounds and mixtures of at least one vinylimidazole compound with at least one compound which is chosen from N-[3-(dimethylamino)propyl]acrylamide, N-[3-(dimethylamino)propyl]methacrylamide, N,N-dimethylaminoethyl(meth)acrylate, N,N-dimethylaminopropyl(meth)acrylate and mixtures thereof,
0.1 to 2% by weight of at least one crosslinker b), preferably ethylene glycol di(meth)acrylate and/or pentaerythritol triallyl ether,
0.05 to 30% by weight, particularly preferably 0.1 to 20% by weight, in particular 0.1 to 15% by weight, of at least one silicone compound c),
20 to 95% by weight of vinylpyrrolidone and/or vinylcaprolactam d),
5 to 40% by weight of at least one monomer f) which is preferably chosen from C₁-C₆-(meth)acrylates, in particular methyl(meth)acrylate, ethyl(meth)acrylate, n-butyl acrylate and mixtures thereof.

Particular preference is also given to the use of an ampholytic copolymer containing silicone groups which are obtainable by free-radical copolymerization of
at least 5% by weight, based on the total weight of the monomers used for the polymerization, of at least one monomer pair of N-vinylimidazole a2) and acrylic acid and/or methacrylic acid a1),
3 to 70% by weight of at least one monomer with a cationogenic or cationic group, preferably chosen from vinylimidazole compounds and mixtures of at least one vinylimidazole compound with at least one compound which is chosen from N-[3-(dimethylamino)propyl]acrylamide, N-[3-(dimethylamino)propyl]methacrylamide, N,N-dimethylaminoethyl(meth)acrylate, N,N-dimethylaminopropyl(meth)acrylate and mixtures thereof,
0.1 to 2% by weight of at least one crosslinker c), preferably ethylene glycol di(meth)acrylate and/or pentaerythritol triallyl ether,
0.05 to 30% by weight, particularly preferably 0.1 to 20% by weight, in particular 0.1 to 15% by weight, of at least one silicone compound c),
20 to 85% by weight of vinylpyrrolidone and/or vinylcaprolactam,
0.5 to 20% by weight, preferably 1 to 10% by weight, of at least one further monomer e) which is chosen from C₈-C₂₂-(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof.

In a specific embodiment, all of the abovementioned cationic copolymers containing silicone groups are subjected to partial or complete quaternization. Suitable quaternizing agents are those specified below. A particularly preferred quaternizing agent is CH₃—Cl.

In a specific embodiment, the free-radical copolymerization of the abovementioned components a1), a2), b) and, if present, d) to g) is carried out in the presence of at least one polyether-containing compound which has no copolymerizable double bond. Here, specific copolymers having advantageous properties are obtained. This can be attributed, for example, to the effect of the polyether component as protective colloid or emulsifier. This can, for example, also result from an at least partial grafting onto the polyether component as graft base. However, mechanisms other than grafting are also conceivable. The copolymers used according to the invention comprise, quite generally, the process products of the free-radical copolymerization, which are understood as meaning, for example, pure graft polymers, mixtures of graft polymers with ungrafted compounds of the polyether component, and any desired mixtures.

Preferably, the amount of polyether component used (if present) is 0.1 to 50% by weight, particularly preferably 1 to 25% by weight, based on the total weight of the components used for the polymerization.

Suitable polyether-containing compounds are, for example, water-soluble or water-dispersible nonionic polymers which have alkylene oxide repeat units. The fraction of alkylene oxide repeat units is preferably at least 30% by weight, based on the total weight of the compound. Suitable polyether-containing compounds are, for example, polyalkylene glycols, as are usually also used as nonionic surfactants. Suitable polyalkylene glycols generally have a number-average molecular weight in the range from about 150 to 100 000, preferably 300 to 50 000, particularly preferably 500 to 40 000. Suitable polyalkylene glycols are, for example, polyethylene glycols, polypropylene glycols, polytetrahydrofurans and alkylene oxide copolymers. Suitable alkylene oxides for the preparation of alkylene oxide copolymers are, for example, ethylene oxide, propylene oxide, epichlorohydrin, 1,2- and 2,3-butylene oxide. The alkylene oxide copolymers can comprise the copolymerized alkylene oxide units in random distribution or in the form of blocks. Advantageously, homopolymers of ethylene oxide or copolymers which comprise ethylene oxide are used. Preferably, the fraction of repeat units derived from ethylene oxide is 40 to 99% by weight. Of suitability are, for example, copolymers of ethylene oxide and propylene oxide, copolymers of ethylene oxide and butylene oxide, and copolymers of ethylene oxide, propylene oxide and at least one butylene oxide.

The copolymers used according to the invention are prepared in accordance with customary processes known to the person skilled in the art, e.g. by solution polymerization, precipitation polymerization, suspension polymerization or emulsion polymerization. The W/W polymerization in water with a suitable displacing agent, e.g. a salt, such as NaCl, is also suitable.

Preferred solvents for the solution polymerization are aqueous solvents, such as water and mixtures of water with water-miscible solvents, for example alcohols, such as methanol, ethanol, n-propanol, isopropanol, n-butanol, sec-butanol, tert-butanol, n-hexanol and cyclohexanol, and glycols, such as ethylene glycol, propylene glycol and butylene glycol, and the methyl or ethyl ethers of the dihydric alcohols, diethylene glycol, triethylene glycol, polyethylene glycols with number-average molecular weights up to about 3000, glycerol and dioxane. Particular preference is given to the polymerization in water or a water/alcohol mixture, for example in a water/ethanol mixture. The polymerization temperatures in the case of solution polymerization are preferably in a range from about 30 to 120° C., particularly preferably 40 to 100° C.

The copolymers are particularly preferably prepared by precipitation polymerization.

The precipitation polymerization is preferably carried out in a largely anhydrous, aprotic solvent or solvent mixture, preferably in ethyl acetate and/or n-butyl acetate. A largely anhydrous, aprotic solvent or solvent mixture is understood as meaning a solvent or solvent mixture with a water content of at most 5% by weight.

The precipitation polymerization preferably takes place at a temperature in the range from 70 to 140° C., preferably 75 to 100° C., in particular from 80 to 95° C. The resulting polymer particles precipitate out of the reaction solution and can be isolated by customary methods, such as filtration using subatmospheric pressure. For the precipitation polymerization it is possible to use surface-active, polymeric compounds, preferably based on polysiloxanes. In the case of precipitation polymerization, the polymers obtained usually have higher molecular weights than those in the case of solution polymerization.

The polymerization is usually carried out under atmospheric pressure, although it can also proceed under reduced or elevated pressure. A suitable pressure range is between 1 and 5 bar.

To prepare the polymers, the monomers can be polymerized with the help of initiators which form free radicals.

Initiators for the free-radical polymerization which can be used are the peroxo and/or azo compounds customary for this purpose, for example alkali metal or ammonium peroxydisulfates, diacetyl peroxide, dibenzoyl peroxide, succinyl peroxide, di-tert-butyl peroxide, tert-butyl perbenzoate, tert-butyl perpivalate, tert-butyl peroxy-2-ethylhexanoate, tert-butyl permaleate, cumene hydroperoxide, diisopropyl peroxydicarbamate, bis-(o-toloyl) peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, azobisisobutyronitrile, azobis(2-amidinopropane)dihydrochloride or 2,2′-azobis(2-methylbutyronitrile). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate, H₂O₂/Cu^{I}.

In a specific embodiment, for the preparation of the copolymers according to the invention, at least two free radical initiators are used which permit an essentially independent initiation in at least two phases. Here, copolymers with particularly low residual monomer contents can be achieved.

Preferably, for the copolymerization, at least two initiators are used whose decomposition temperatures are different from one another by at least 10° C. Within the scope of the invention, the decomposition temperature is defined as the temperature at which 50% of the molecules decompose into free radicals within 2.5 hours. Preferably, in the case of this procedure, the copolymerization takes place until conclusion of the precipitation of the copolymer at a temperature greater than or equal to the lower decomposition temperature and less than the higher decomposition temperature, and, after the precipitation, a further reaction takes place at a temperature greater than or equal to the higher decomposition temperature.

Preferably, the method according to the invention comprises a first polymerization phase at a first polymerization temperature and a second polymerization phase at a second polymerization temperature above the first polymerization temperature, where, for the polymerization, at least two initiators are used whose half-lives at the first polymerization temperature differ in such a way that at least one of these initiators decomposes into free radicals during the first polymerization phase and at least one of these initiators essentially does not decompose into free radicals during the first polymerization phase and decomposes into free radicals during the second polymerization phase. Preferably, in the case of this procedure, the second polymerization phase starts essentially after precipitation of the copolymer. “essentially” after precipitation of the copolymer is understood as meaning that the copolymer is preferably present to at least 80% by weight, preferably at least 90% by weight, in particular at least 95% by weight, based on the total weight of the copolymer, in precipitated form.

The half-life of an initiator can be determined by customary methods known to the person skilled in the art, as described, for example, in the publication “Initiators for high polymers”, Akzo Nobel, No. 10737. Preferably, the half-life of the first polymerization initiator at the first polymerization temperature and of the second polymerization initiator at the second polymerization temperature is in a range from about 1 minute to 3 hours, particularly preferably 5 minutes to 2.5 hours. If desired, shorter half-lives of, for example, 1 second to 1 minute, or longer half-lives than 3 hours can also be used provided it is ensured that the initiator(s) decomposing at the higher temperature essentially decomposes into free radicals during the second polymerization phase.

In addition to the first and second polymerization phase, further polymerization phases at polymerization temperatures different therefrom can be used. Thus, it is, for example, possible to carry out a first polymerization phase at a first polymerization temperature which is chosen such that controlled polymerization (i.e. e.g. with avoidance of an undesired temperature increase as a result of the heat of reaction, of an excessively high reaction rate, etc.) takes place. An afterpolymerization can then, for example, follow at a temperature which is above the first polymerization temperature but below the second and which is chosen so that the initiator(s) decomposing at the higher temperature do not essentially decompose into free radicals. Following completion of this afterpolymerization, to which, if desired, the initiator decomposing at the lower temperature and/or another initiator decomposing under the conditions of the afterpolymerization can also be added, the second polymerization phase can then follow.

Preferably, the initiator system used comprises at least two initiators whose decomposition temperatures differ from one another by at least 15° C.

The initiator decomposing at the lower temperature preferably has a decomposition temperature of from 50 to 100° C.

The initiator decomposing at the higher temperature preferably has a decomposition temperature of from 80 to 150° C.

Preferably, the initiator decomposing at the higher temperature is initially introduced at the start of the copolymerization or is added before or during the precipitation of the copolymer.

Preferably, the initiator decomposing at the higher temperature is initially introduced at the start of the copolymerization or is added before the precipitation of the copolymer.

In a preferred initiator combination, the initiator decomposing at the lower temperature is Trigonox® EHP (bis(2-ethylhexyl)peroxydicarbonate, CAS-No. 16111-62-9) and the initiator decomposing at the higher temperature is chosen from tert-butyl peroxypivalate (e.g. Luperox 11 M75 from Atochem), tert-butyl peroctoate, lauroyl peroxide (LPO, CAS-No. 105-74-8) or 2,5-dimethyl-2,5-bis(t-butylperoxy)hexane (Trigonox® 101).

A further preferred initiator combination comprises Trigonox® EHP and tert-butyl peroctoate.

A further preferred initiator combination comprises lauroyl peroxide and tert-butyl peroctoate or 2,5-dimethyl-2,5-bis(t-butylperoxy)hexane (Trigonox® 101).

A further preferred initiator combination comprises tert-butyl peroxypivalate (Luperox 11 M75 and tert-butyl peroctoate or 2,5-dimethyl-2,5-bis(t-butylperoxy)hexane (Trigonox® 101).

A further preferred initiator combination comprises tert-butyl peroctoate and 2,5-dimethyl-2,5-bis(t-butylperoxy)hexane (Trigonox® 101).

To achieve the purest polymers possible, the polymers can, for example, be subjected to a washing step with a suitable solvent, e.g. the solvent also used for the polymerization.

The copolymers can be converted into a solid form, in particular a powder, by various drying processes. Polymers obtained by precipitation polymerization are preferably isolated by filtration prior to drying. Preference is given to using vacuum drying processes, such as vacuum fluidized-bed drying. The solid copolymer compositions obtained in this way can advantageously be dissolved or redispersed again in aqueous media. Suitable aqueous media are water and mixtures of water with at least one water-miscible solvent. Preference is given to water and water/alcohol mixtures, where the alcohol is preferably chosen from C₁-C₄-alkanols, such as ethanol, isopropanol, n-butanol and tert-butanol. Solid copolymers have the advantage of better storability, easier transportability and generally exhibit a lower tendency for microbial attack.

The anionogenic groups (acid groups) of the copolymers can be partially or completely neutralized with a base. Bases which can be used for the neutralization of the polymers are alkali metal bases such as sodium hydroxide solution, potassium hydroxide solution, sodium carbonate, sodium hydrogencarbonate, potassium carbonate or potassium hydrogencarbonate and alkaline earth metal bases, such as calcium hydroxide, calcium oxide, magnesium hydroxide or magnesium carbonate, and amines. Suitable amines are, for example, C₁-C₆-alkylamines, preferably n-propylamine and n-butylamine, dialkylamines, preferably diethylpropylamine and dipropylmethylamine, trialkylamines, preferably triethylamine and triisopropylamine. Preference is given to amino alcohols, e.g. trialkanolamines, such as triethanolamine, alkyldialkanolamines, such as methyl- or ethyidiethanolamine and dialkylalkanolamines, such as dimethylethanolamine, and 2-amino-2-methyl-1-propanol. For use in hair-treatment compositions, NaOH, KOH, 2-amino-2-methyl-1-propanol, 2-amino-2-ethylpropane-1,3-diol, diethylaminopropylamine and triisopropanolamine have proven useful for neutralizing the polymers comprising acid groups. The neutralization of the acid groups can also be carried out using mixtures of two or more bases, e.g. mixtures of sodium hydroxide solution and triisopropanolamine. Depending on the intended use, the neutralization can be carried out partially or completely. Partial neutralization is preferably understood as meaning neutralization of up to 95%, preferably up to 50%, specifically up to 25%, of the acid groups present in the polymer. Furthermore, partial neutralization is preferably understood as meaning neutralization of at least 1%, preferably at least 5%, of the acid groups present in the polymer.

Charged cationic groups can be produced from the present cationogenic nitrogen-containing groups either by protonation, e.g. with mono- or polybasic carboxylic acids, such as lactic acid or tartaric acid, or with mineral acids, such as phosphoric acid, sulfuric acid and hydrochloric acid, or by quaternization, e.g. with alkylating agents, such as C₁-C₄-alkyl halides or sulfates. Examples of such alkylating agents are ethyl chloride, ethyl bromide, methyl chloride, methyl bromide, dimethyl sulfate and diethyl sulfate.

If the copolymers used according to the invention are to be both quaternized and also neutralized, then the quaternization is preferably carried out first, followed by the neutralization.

The copolymers described above are advantageously suitable for modifying the rheological properties of hair cosmetic compositions. In particular, these are aqueous compositions. In this connection, the compositions of the copolymers are advantageously generally clear. The hair cosmetic formulations can thus be colored without impairment by the intrinsic color of the compositions. Furthermore, the compositions can be formulated in the form of opaque to clear gels.

The anionically ampholytic copolymers used according to the invention are specifically suitable as rheology modifiers with properties which can be controlled via the pH. They are specifically suitable as pH-switchable thickeners for a pH range greater than or equal to 6.

The quaternized cationic copolymers used according to the invention have a high pH stability in a pH range from about 3 to 9. The (partially) neutralized copolymers used according to the invention are specifically suitable as rheology modifiers with properties which can be controlled via the pH. They are specifically suitable as pH-switchable thickeners for a pH range from about 4 to 7.

The anionically ampholytic copolymers and cationically ampholytic copolymers used according to the invention are also specifically suitable as rheology modifiers for salt-containing compositions. On the other hand, it is also possible to thicken salt-free compositions using the copolymers according to the invention.

Advantageously, the anionically ampholytic copolymers and cationically ampholytic copolymers used according to the invention also act as film-forming and/or conditioning rheology modifiers. They are thus suitable specifically for hair-setting compositions as “setting thickeners” and in haircare compositions as “conditioning thickeners”.

The copolymers are suitable both for the preparation of homogeneous-phase aqueous compositions, and also for the formulation of heterogeneous-phase compositions which additionally comprise at least one water-insoluble (hydrophobic) liquid or solid compound. “Homogeneous-phase compositions” have only a single phase irrespective of the number of their constituents. “Heterogeneous-phase compositions” are disperse systems of two or more immiscible components. These include solid/liquid, liquid/liquid and solid/liquid/liquid compositions, such as dispersions and emulsions, e.g. O/M and W/O formulations which have at least one of the oil or fat components described in more detail below and water as immiscible phases. In principle, the copolymers can be used either in the water phase or in the oil phase. In general, heterogeneous-phase liquid/liquid compositions comprise the copolymers essentially in the water phase.

The invention further provides a hair cosmetic composition comprising
A) at least one ampholytic copolymer as defined above,
B) if appropriate at least one hair polymer different from A),
C) at least one cosmetically acceptable carrier, and
D) if appropriate at least one cosmetically acceptable active ingredient and/or auxiliary different from A) and B).

The ampholytic copolymers A) used according to the invention are advantageously characterized not only by good thickening properties, but also by film-forming properties. They can thus be used in hair cosmetic compositions also as hair-setting component, meaning that the use of additional setting polymers B) is only required in a reduced amount or may even be entirely superfluous. The ampholytic copolymers A) are also advantageously characterized by conditioning properties and can improve the sensory properties of the hair, e.g. give it suppleness and shine. Hair treated with the copolymers A) are not sticky or are only very slightly sticky.

The anionically ampholytic copolymers A) used according to the invention are characterized by their good application properties in the pH range from 6 to 10, preferably 6.5 to 8.

The cationically ampholytic copolymers A) used according to the invention are characterized by their good application properties in the pH range from 4 to 9.

The cosmetic compositions comprise the polymer component A) preferably in an amount of from about 0.1 to 10% by weight, particularly preferably 0.2 to 6% by weight, in particular 0.3 to 3% by weight, based on the total weight of the composition.

Suitable hair polymers B) are quite generally film-forming polymers which are cosmetically compatible and are used for formulating hair cosmetic compositions. They are preferably water-soluble or water-dispersible polymers. Particular preference is given to water-soluble hair polymers. These include, quite generally, anionic, cationic, amphoteric and nonionic polymers which are different from the copolymers A).

Examples of anionic polymers are homopolymers and copolymers of acrylic acid and methacrylic acid or salts thereof, copolymers of acrylic acid and acrylamide and salts thereof; sodium salts of polyhydroxycarboxylic acids, polyaspartic acid and salts and derivatives thereof, water-soluble or water-dispersible polyesters, polyurethanes, e.g. Luviset PUR® from BASF, and polyureas. Particularly suitable polymers are copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid and if appropriate further vinyl esters (e.g. Luviset® grades), maleic anhydride copolymers, if appropriate reacted with alcohol, anionic polysiloxanes, e.g. carboxyfunctional ones, t-butyl acrylate, methacrylic acid (e.g. Luviskol® VBM), copolymers of acrylic acid and methacrylic acid with hydrophobic monomers, such as, for example, C₄-C₃₀-alkyl esters of (meth)acrylic acid, C₄-C₃₀-alkylvinyl esters, C₄-C₃₀-alkyl vinyl ethers and hyaluronic acid. Examples of anionic polymers are also vinyl acetate/crotonic acid copolymers, as are sold, for example, under the names Resyn® (National Starch) and Gafset® (GAF), and vinylpyrrolidone/vinyl acrylate copolymers obtainable, for example, under the trade name Luviflex® (BASF). Further suitable polymers are the vinylpyrrolidone/acrylate terpolymer obtainable under the name Luviflex® VBM-35 (BASF) and polyamides containing sodium sulfonate or polyesters containing sodium sulfonate. Also suitable are vinylpyrrolidone/ethyl methacrylate/methacrylic acid copolymers, as are sold by Stepan under the names Stepanhold-Extra and -R1, and the Carboset® grades from BF Goodrich.

Suitable cationic polymers are, for example, cationic polymers with the INCI name Polyquaternium, e.g. copolymers of vinylpyrrolidone/N-vinylimidazolium salts (Luviquat® FC, Luviquat® HM, Luviquat® MS, Luviset Clear®, Luviquat Supreme®, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamido copolymers (Polyquaternium-7) and chitosan. Suitable cationic polymers are also polyethyleneimines and salts thereof and polyvinylamines and salts thereof. Suitable cationic (quaternized) polymers are also Merquat® (polymer based on dimethyldiallylammonium chloride), Gafquat® (quaternary polymers which are formed by reacting polyvinylpyrrolidone with quaternary ammonium compounds), Polymer JR (hydroxyethylcellulose with cationic groups) and cationic polymers based on plants, e.g. guar polymers, such as the Jaguar® grades from Rhodia.

Very particularly suitable polymers are nonionic polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, and nonionic cellulose derivatives. These include, for example, Luviflex® Swing (partially saponified copolymer of polyvinyl acetate and polyethylene glycol, BASF).

Suitable polymers are also nonionic, water-soluble or water-dispersible polymers or oligomers, such as polyvinylcaprolactam, e.g. Luviskol® Plus (BASF), or polyvinylpyrrolidone and copolymers thereof, in particular with vinyl esters, such as vinyl acetate, e.g. Luviskol® VA 37 (BASF); polyamides, e.g. based on itaconic acid and aliphatic diamines, as are described, for example, in DE-A-43 33 238.

Suitable polymers are also amphoteric or zwitterionic polymers, such as the octylacrylamide/methyl methacrylate/tert-butylaminoethyl methacrylate/2-hydroxypropyl methacrylate copolymers obtainable under the names Amphomer® (National Starch), and zwitterionic polymers, as are disclosed, for example, in the German patent applications DE 39 29 973, DE 21 50 557, DE 28 17 369 and DE 37 08 451. Acrylamidopropyltrimethylammonium chloride/acrylic acid or methacrylic acid copolymers and alkali metal and ammonium salts thereof are preferred zwitterionic polymers. Further suitable zwitterionic polymers are methacroylethylbetaine/methacrylate copolymers, which are commercially available under the name Amersette® (AMERCHOL), and copolymers of hydroxyethyl methacrylate, methyl methacrylate, N,N-dimethylaminoethyl methacrylate and acrylic acid (Jordapon®).

Suitable polymers are also nonionic, siloxane-containing, water-soluble or water-dispersible polymers, e.g. polyether siloxanes, such as Tegopren® (Goldschmidt) or Belsil® (Wacker).

The cosmetic compositions comprise the hair polymers B) preferably in an amount of from about 0.001 to 30% by weight, particularly preferably 0.01 to 15% by weight, in particular 0.1 to 7% by weight, based on the total weight of the composition. The compositions preferably have a carrier component C) which is chosen from water, hydrophilic components, hydrophobic components and mixtures thereof.

Suitable hydrophilic carriers C) are, for example, water, mono-, di- or polyhydric alcohols having preferably 1 to 8 carbon atoms, such as ethanol, n-propanol, isopropanol, propylene glycol, glycerol, sorbitol, etc. Preferred hydrophilic carriers are water and mixtures of water and at least one C₂-C₆-alkanol, such as ethanol, n-propanol and isopropanol.

Suitable hydrophobic carriers C) are preferably chosen from
i) oils, fats, waxes,
ii) esters of C₆-C₃₀-monocarboxylic acids with mono-, di- or trihydric alcohols which are different from iii),
iii) saturated acyclic and cyclic hydrocarbons,
iv) fatty acids,
v) fatty alcohols,
vi) propellant gases,
   and mixtures thereof.

Suitable silicone oils C) are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. Preferred cyclic siloxanes have 4- to 8-membered rings. Suitable cyclic siloxanes are commercially available, for example, under the name cyclomethicone.

Preferred oil and fat components C) are chosen from paraffin and paraffin oils; vaseline; natural fats and oils, such as castor oil, soya oil, peanut oil, olive oil, sunflower oil, sesame oil, avocado oil, cocoa butter, almond oil, peach kernel oil, ricinus oil, cod-liver oil, pig fat, spermaceti, spermaceti oil, sperm oil, wheatgerm oil, macadamia nut oil, evening primrose oil, jojoba oil; fatty alcohols, such as lauryl alcohol, myristyl alcohol, cetyl alcohol, stearyl alcohol, oleyl alcohol, cetyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candililla wax, spermaceti, and mixtures of the abovementioned oil and fat components.

Suitable cosmetically and pharmaceutically compatible oil and fat components C) are described in Karl-Heinz Schrader, Grundlagen und Rezepturen der Kosmetika [Fundamentals and formulations of cosmetics], 2nd edition, Verlag Hüthig, Heidelberg, pp. 319-355, which is hereby incorporated by reference.

Preferred carriers C) are also propellants. Suitable propellants C) are those customarily used for hair sprays or aerosol foams. Preference is given to mixtures of propane/butane, pentane, dimethyl ether, 1,1-difluoroethane (HFC-152 a), carbon dioxide, nitrogen or compressed air.

In addition, the compositions according to the invention can comprise, as component D), at least one further cosmetic active ingredient or auxiliary different from A) and B).

The components D) are chosen according to the desired field of use of the composition. Besides components which are typical of the field of use, they are preferably chosen from cosmetically active ingredients, excipients, emulsifiers, surfactants, preservatives, fragrances (e.g. perfume oils), thickeners different from component A), polymers different from component A) (e.g. hair polymers, hair conditioners, graft polymers, water-soluble or dispersible silicone-containing polymers), gel formers, dyes, pigments, photoprotective agents, consistency regulators, antioxidants, bleaches, care agents, tints, tanning agents, humectants, refatting agents, collagen, protein hydrolysates, lipids, emollients, softeners, antifoams, antistats, resins, solvents, solubility promoters, neutralizing agents, stabilizers, sterilizing agents, propellants, drying agents, opacifiers, etc.

The copolymers A) can be used together with conventional thickeners D). Suitable conventional thickeners are crosslinked polyacrylic acids and derivatives thereof, polysaccharides and derivatives thereof, such as xanthan gum, agar agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone. Preferred thickeners are compounds which impart a pseudoplastic flow behavior to the formulation, i.e. high viscosity in the stationary state and low viscosity in the agitated state. Particular preference is given to Xanthan Gum® (Kelzan® from Kelco), Rhodopol® 23 (Rhone Poulenc), Veegum® (R. T. Vanderbilt) or Attaclay® (Engelhardt).

Preferably, the compositions according to the invention are in the form of a gel, foam, spray, ointment, cream, emulsion, suspension, lotion, milk or paste. If desired, liposomes or microspheres can also be used.

On account of their thickening and film-forming properties, the copolymers A) described above are suitable in particular as additives for formulation in the form of gels.

Preferred cosmetically active ingredients are, for example, bleaches, keratin-hardening substances, antimicrobial active ingredients, photofilter active ingredients, repellent active ingredients, hyperemic substances, keratolytic and keratoplastic substances, antidandruff active ingredients, antiphlogistics, keratinizing substances, active substances which act as antioxidants and/or as free-radical scavengers, deodorizing active ingredients, sebostatic active ingredients, plant extracts, antierythimatous or antiallergic active ingredients and mixtures thereof.

Suitable keratin-hardening substances are generally active substances as are also used in antiperspirants, such as, for example, potassium aluminum sulfate, aluminum hydroxychloride, aluminum lactate, etc. Antimicrobial active substances are used in order to destroy microorganisms and/or to inhibit their growth and thus serve both as preservatives and also as deodorizing substance which reduces the formation or the intensity of body odor. These include, for example, customary preservatives known to the person skilled in the art, such as p-hydroxybenzoates, imidazolidinylurea, formaldehyde, sorbic acid, benzoic acid, salicylic acid, etc. Such deodorizing substances are, for example, zinc ricinoleate, triclosan, undecylenic acid alkylolamides, triethyl citrate, chlorhexidine etc. Suitable photofilter active substances are substances which absorb UV rays in the UV-B and/or UV-A region. Suitable UV filters are those specified above. Also suitable are p-aminobenzoic esters, cinnamic esters, benzophenones, camphor derivatives, and pigments which stop UV rays, such as titanium dioxide, talc and zinc oxide. Suitable repellant active substances are compounds which are able to keep or drive certain animals, in particular insects, away from people. These include, for example, 2-ethyl-1,3-hexanediol, N,N-diethyl-m-toluamide etc. Suitable hyperemic substances, which stimulate blood flow in the skin, are, for example, essential oils, such as dwarf-pine, lavender, rosemary, juniper berry, horse chestnut extract, birch leaf extract, hay flower extract, ethyl acetate, camphor, menthol, peppermint oil, rosemary extract, eucalyptus oil, etc. Suitable keratolytic and keratoplastic substances are, for example, salicylic acid, calcium thioglycolate, thioglycolic acid and its salts, sulfur, etc. Suitable antidandruff active substances are, for example, sulfur, sulfur polyethylene glycol sorbitan monooleate, sulfur ricinol polyethoxylate, zinc pyrithione, aluminum pyrithione, etc. Suitable antiphlogistics, which counteract skin irritations, are, for example, allantoin, bisabolol, dragosantol, camomile extract, panthenol, etc.

Preferably, the hair-treatment compositions according to the invention are in the form of a setting foam, hair mousse, hair gel, hair wax, shampoo, hair spray, hair foam, end fluids, neutralizers for permanent waves or hot oil treatments, in particular in the form of a hair gel, hair wax or shampoo. Depending on the field of use, the hair cosmetic preparations can be applied in the form of (aerosol) spray, (aerosol) foam, gel, gel spray, cream, lotion or wax. Hair sprays here comprise both aerosol sprays and also pump sprays without propellant gas. Hair foams comprise both aerosol foams and pump foams without propellant gas. Hair sprays and hair foams comprise preferably predominantly or exclusively water-soluble or water-dispersible components. If the compounds used in the hair sprays and hair foams according to the invention are water-dispersible, they can be used in the form of aqueous microdispersions having particle diameters of usually 1 to 350 nm, preferably 1 to 250 nm. The solids contents of these preparations are usually in a range from about 0.5 to 20% by weight. These microdispersions generally require no emulsifiers or surfactants for their stabilization.

The hair cosmetic formulations according to the invention comprise, in a preferred embodiment,
0.05 to 5% by weight of at least one copolymer A),
0 to 7% by weight, e.g. 0.01 to 5% by weight, of at least one hair-setting polymer B),
83 to 99.95% by weight of at least one carrier chosen from water and water/alcohol mixtures which comprise up to 20% by weight of alcohol, based on the weight of the water/alcohol mixture, and also propellants,
0 to 5% by weight, preferably 0.01 to 3% by weight of at least one further constituent.

Alcohol is understood as meaning all alcohols customary in cosmetics, e.g. ethanol, isopropanol, n-propanol.

Further constituents are understood as meaning the additives customary in cosmetics, for example antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active substances, UV filters, care substances, such as panthenol, collagen, vitamins, protein hydrolysates, alpha- and beta-hydroxycarboxylic acids, stabilizers, pH regulators, dyes, viscosity regulators, gel formers, salts, humectants, refatting agents, complex formers and further customary additives.

Also included here are all styling and conditioner polymers known in cosmetics which can be used in combination with the polymers according to the invention if very specific properties are to be set.

Suitable conventional hair cosmetic polymers are, for example, the abovementioned cationic, anionic, neutral, nonionic and amphoteric polymers, which are hereby incorporated by reference.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyethersiloxanes, silicone resins or dimethicone copolyols (CTFA) and amino-functional silicone compounds such as amodimethicones (CTFA).

Emulsifiers which may be used are all of the emulsifiers customarily used in hair foams. Suitable emulsifiers may be nonionic, cationic or anionic or amphoteric.

Examples of nonionic emulsifiers (INCI nomenclature) are laureths, e.g. laureth-4; ceteths, e.g. ceteth-1, polyethylene glycol cetyl ether; ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methylsulfate, quaternium-1 to x (INCI).

Anionic emulsifiers may be chosen, for example, from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition:
a) 0.1 to 5% by weight of at least one copolymer A),
b) 0 to 5% by weight of at least one cosmetically acceptable water-soluble or water-dispersible hair-setting polymer B) which is different from A),
c) 80 to 99.85% by weight of water and/or alcohol,
d) 0 to 1% by weight of a gel former different from A),
e) 0 to 20% by weight of further constituents.

Additional gel formers which can be used are all gel formers customary in cosmetics. These include slightly crosslinked polyacrylic acid, for example Carbomer (INCI), cellulose derivatives, e.g. hydroxypropylcellulose, hydroxyethylcellulose, cationically modified celluloses, polysaccharides, e.g. xanthan gum, caprylic/capric triglyceride, sodium acrylate copolymers, polyquaternium-32 (and) Paraffinum Liquidum (INCI), sodium acrylate copolymers (and) Paraffinum Liquidum (and) PPG-1 trideceth-6, acrylamidopropyltrimonium chloride/acrylamide copolymers, steareth-10 allyl ether acrylate copolymers, polyquaternium-37 (and) Paraffinum Liquidum (and) PPG-1 trideceth-6, polyquaternium 37 (and) propylene glycol dicaprate dicaprylate (and) PPG-1 trideceth-6, polyquaternium-7, polyquaternium-44. Crosslinked homopolymers of acrylic acid which are suitable as additional gel formers are commercially available, for example, under the name Carbopol® from BF GOODRICH. Preference is also given to hydrophobically modified crosslinked polyacrylate polymers, such as Carbopol® Ultrez 21 from Noveon. Further examples of anionic polymers which are suitable as gel formers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers, which are available under the name Aculyn® from Rohm and Haas.

The copolymers A) according to the invention can be used in hair cosmetic preparations as conditioners.

According to a further preferred embodiment, the compositions according to the invention are a shampoo formulation.

Such formulations comprise at least one copolymer and usually anionic surfactants as base surfactants and amphoteric and/or nonionic surfactants as cosurfactants. Further suitable active ingredients and/or auxiliaries are generally chosen from lipids, perfume oils, dyes, organic acids, preservatives and antioxidants, and also thickeners/gel formers, skin conditioning agents and humectants.

These formulations preferably comprise 2 to 50% by weight, preferably 5 to 40% by weight, particularly preferably 8 to 30% by weight, of surfactants, based on the total weight of the formulation.

In the shampoo preparations, all of the anionic, neutral, amphoteric or cationic surfactants customarily used in body-cleansing compositions can be used.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

These include, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or -propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 moles per mole of alcohol. In addition, alkylamine oxides, mono- or dialkylalkanolamides, fatty acid esters of polyethylene glycols, ethoxylated fatty acid amides, alkyl polyglycosides or sorbitan ether esters are suitable.

Furthermore, the shampoos can comprise customary cationic surfactants, such as, for example, quaternary ammonium compounds, for example cetyltrimethylammonium chloride.

In addition, the shampoo formulations can comprise thickeners, such as, for example, sodium chloride, PEG-55, propylene glycol oleate, PEG-120 methylglucose dioleate and others, and also preservatives, further active ingredients and auxiliaries and water.

In the shampoo formulations, in order to achieve certain effects, customary conditioners can be used in combination with the copolymers A). These include, for example, the abovementioned cationic polymers with the INCI name Polyquaternium, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamide copolymers (Polyquaternium-7). It is also possible to use protein hydrolysates, and conditioning substances based on silicone compounds, for example polyalkylsiloxanes, polyarylsiloxanes, polyarylalkylsiloxanes, polyethersiloxanes or silicone resins. Further suitable silicone compounds are dimethicone copolyols (CTFA) and aminofunctional silicone compounds, such as amodimethicone (CTFA). In addition, cationic guar derivatives such as guar hydroxypropyltrimonium chloride (INCI) can be used.

The copolymers used according to the invention are preferably suitable for shampoos which comprise no or only small amounts of inorganic salts, specifically alkali metal and alkaline earth metal chlorides, such as NaCl. They are thus advantageously suitable for the preparation of mild shampoos and baby shampoos.

The invention further provides a method for cosmetically treating the hair in which a hair cosmetic composition, as defined above, is prepared and applied to the hair to be treated, if appropriate with the aid of the hands, combs, brushes etc. and/or of water, and if appropriate under the subsequent effect of warm air. In a specific embodiment of this method, the hair, following application and, if appropriate, an action time, is subjected to a washing-out operation (e.g. in the case of compositions in the form of a shampoo). In a second embodiment, the compositions remain on the hair following application as long as the hair cosmetic effect associated with them persists or is desired and are only then removed by washing out.

The invention is illustrated in more detail by reference to the following nonlimiting examples.

General Preparation Procedure A1 (Anionically Ampholytic Copolymers):

### EXAMPLE 23

### Copolymer of VP/MAA/VI/MMA/C₁₆-₁₈-PEG-MA/EGDMA

| | | | |
|---|---|---|---|
| Initial charge: | 412 | g | butyl acetate |
| Feed 1: | 58.5 | g | vinylpyrrolidone |
| | 9.0 | g | vinylimidazole |
| | 66.0 | g | methacrylic acid |
| | 15.0 | g | Plex-6877 O ® (25% strength |
| | | | C₁₆-C₁₈-PEG-MA in methyl |
| | | | methacrylate) |
| | 1.5 | g | ethylene glycol dimethacrylate |
| Feed 2 | 38.2 | g | butyl acetate |
| | 0.15 | g | tert-butyl peroctoate |
| Feed 3: | 95.6 | g | butyl acetate |
| | 0.39 | g | tert-butyl peroctoate |
| Feed 4: | 15 | g | methyl chloride (about 1.5 times the weight |
| | | | of vinylimidazole) |
| Feed 5: | 23 | g | triethanolamine (about 20% based on |
| | | | methacrylic acid) |

At 85 to 88° C., feed 1 and feed 2 were added to a stirred apparatus fitted with reflux condenser, internal thermometer and four feed devices over the course of two hours. The reaction mixture was then stirred for a further 2 h at about 88° C. Feed 3 was then metered in over 30 minutes and the mixture was after-polymerized at 90° C. for 3 hours. The product which precipitated out in the form of a white powder was quaternized with methyl chloride (feed 4) at a temperature between 70 and 100° C. After cooling to about 40° C., the product was partially neutralized with triethanolamine (feed 5) at 40° C. over 1 h. The powder was removed using a suction filter, washed at least twice with acetone and dried under reduced pressure at 40° C.

To prepare the polymers 26 to 40, Belsil® DMC 6031, Wacker, was introduced in the initial charge.

The polymers 2, 5, 7, 9, 13 to 17, 20, 24, 25,26, 31, 32, 34, 35, 36, 38, 39 and 40 given in Table I were prepared in an analogous manner.

The polymers 3, 4, 6, 8, 10, 11, 12, 27 to 30 and 33 were prepared analogously to the preparation procedure A, except that the partial neutralization with triethanolamine was omitted.

The polymers 1, 18, 19, 21, 22 and 37 were prepared analogously to the preparation procedure A1, except that the quaternization with methyl chloride was omitted.

TABLE I
VP/MAA/VI/MMA/C₁₆₋C₁₈-PEGMA/EGDMA precipitation polymers

| | VP | VCap | MAA | | | SMA | | | | | | | |
|---|---|---|---|---|---|---|---|---|---|---|---|---|---|
| Exam- | [% | [% | [% | VI | | [% | MMA^{#} | C₁₆-₁₈- | ODVE | | | | Neutralization |
| ple | by | by | by | [% by | DMAEMA | by | [% by | PEG-MA^{#} | [% by | EGDMA | Belsil ® | Quaternization | with |
| No. | wt.] | wt.] | wt.] | wt.] | [% by wt.] | wt.] | wt.] | [% by wt.] | wt.] | [% by wt.] | 6031**) | with CH₃—Cl | TEA, DN % |
| 1 | 40 | — | 54 | 5 | — | — | — | — | — | 1.0 | — | — | 20 |
| 2 | 43.7 | — | 45 | 10 | — | — | — | — | — | 1.3 | — | ≧70% | 20 |
| 3 | 63.7 | — | 30 | 5 | — | — | — | — | — | 1.3 | — | ≧70% | — |
| 4 | 73.7 | — | 20 | 5 | — | — | — | — | — | 1.3 | — | ≧70% | — |
| 5 | 33.6 | 10 | 45 | 10 | — | — | — | — | — | 1.4 | — | ≧70%*) | 20 |
| 6 | 23.6 | 10 | 50 | 15 | — | — | — | — | — | 1.4 | — | ≧70% | — |
| 7 | 43.8 | 10 | 35 | 10 | — | — | — | — | — | 1.2 | — | ≧70%*) | 20 |
| 8 | 53.8 | 10 | 27 | 8 | — | — | — | — | — | 1.2 | — | ≧70% | — |
| 9 | 43.7 | — | 45 | 10 | — | — | — | — | — | 1.3 | — | ≧70%*) | 20 |
| 10 | 33.6 | — | 50 | 15 | — | — | — | — | — | 1.4 | — | ≧70% | — |
| 11 | 18.5 | — | 60 | 20 | — | — | — | — | — | 1.5 | — | ≧70% | — |
| 12 | 30 | — | 58.8 | 10 | — | — | — | — | — | 1.2 | — | ≧70% | — |
| 13 | 30 | — | 56 | 10 | — | 2.8 | — | — | — | 1.2 | — | ≧70%*) | 20 |
| 14 | 44 | — | 43 | 10 | — | 2 | — | — | — | 1.0 | — | ≧70%*) | 20 |
| 15 | 40 | — | 43 | 13 | — | 3 | — | — | — | 1.0 | — | ≧70%*) | 20 |
| 16 | 38.7 | — | 45 | 10 | — | — | 5 | — | — | 1.3 | — | ≧70%*) | 20 |
| 17 | 33.7 | — | 45 | 10 | — | — | 10 | — | — | 1.3 | — | ≧70%*) | 20 |
| 18 | 39 | — | 51.8 | 3 | — | — | 5 | — | — | 1.2 | — | — | 20 |
| 19 | 39 | — | 45 | 5 | — | — | 10 | — | — | 1.0 | — | — | 20 |
| 20 | 28 | — | 45 | 10 | — | — | 15.7 | — | — | 1.3 | — | ≧70%*) | 20 |
| 21 | 35 | — | 54 | 5 | — | — | 3.75 | 1.25 | — | 1.0 | — | — | 20 |
| 22 | 40 | — | 51 | 3 | — | — | 3.75 | 1.25 | — | 1.0 | — | — | 20 |
| 23 | 39 | — | 44 | 6 | — | — | 7.5 | 2.5 | — | 1.0 | — | ≧70%*) | 20 |
| 24 | 39 | — | 46 | 10 | — | — | — | — | 4 | 1.0 | — | ≧70%*) | 20 |
| 25 | 40 | — | 42 | 14 | — | — | — | — | 3 | 1.0 | — | ≧70%*) | 20 |
| 26 | 43.7 | — | 45 | 10 | — | — | — | — | — | 1.3 | 1 | ≧70%*) | 20 |
| 27 | 33.6 | — | 50 | 15 | — | — | — | — | — | 1.4 | 0.5 | ≧70% | — |
| 28 | 18.5 | — | 60 | 20 | — | — | — | — | — | 1.5 | 2 | ≧70% | — |
| 29 | 30 | — | 58.8 | 10 | — | — | — | — | — | 1.2 | 1 | ≧70% | — |
| 30 | 23.6 | 10 | 50 | 15 | — | — | — | — | — | 1.4 | 1 | ≧70% | — |
| 31 | 33.6 | 10 | 45 | 10 | — | — | — | — | — | 1.4 | 1 | ≧70%*) | 20 |
| 32 | 40.8 | 10 | 40 | 8 | — | — | — | — | — | 1.2 | 0.5 | ≧70%*) | 20 |
| 33 | 50.8 | 10 | 30 | 8 | — | — | — | — | — | 1.2 | 0.5 | ≧70% | — |
| 34 | 40 | — | 43 | 13 | — | 3 | — | — | — | 1.0 | 0.5 | ≧70%*) | 20 |
| 35 | 38.7 | — | 45 | 10 | — | — | 5 | — | — | 1.3 | 0.5 | ≧70%*) | 20 |
| 36 | 33.7 | — | 45 | 10 | — | — | 10 | — | — | 1.3 | 1 | ≧70%*) | 20 |
| 37 | 39 | — | 50 | 4.7 | — | — | 5 | — | — | 1.3 | 0.5 | — | 20 |
| 38 | 33 | — | 50 | 5.7 | — | — | 10 | — | — | 1.3 | 0.5 | ≧70%*) | 20 |
| 39 | 39 | — | 44 | 6 | — | — | 7.5 | 2.5 | — | 1.0 | 0.5 | ≧70%*) | 20 |
| 40 | 40 | — | 42 | 14 | — | — | — | — | 3 | 1.0 | 0.5 | ≧70%*) | 20 |

VP = N-vinylpyrrolidone
VCap = N-vinylcaprolactam
MAA = methacrylic acid
VI = N-vinylimidazole
DMAEMA = N,N-dimethylaminoethyl methacrylate
SMA = stearyl methacrylate
MMA = methyl methacrylate
C₁₆₋C₁₈-PEG-MA = polyethylene glycol methacrylate terminated with a C₁₆₋C₁₈ fatty alcohol mixture
ODVE octadecyl vinyl ether
EGDMA ethylene glycol dimethacrylate
TEA/DN % triethanolamine/degree of neutralization
*)The product was partially neutralized with triethanolamine after the quaternization.
^{#}MMA and C₁₆₋₁₈-PEGMA can be used together, e.g. in the form of the commercial product Plex-6877 O ®, Degussa, Germany.
**)Belsil ® 6031 = ethoxylated/propoxylated dimethylsiloxane, Wacker, Germany, stated in %, based on 100% by weight of the total monomers

General Preparation Procedure A2 (Cationically Ampholytic Copolymers):

### EXAMPLE 61

### Copolymer of VP/MAA/VI/MMA/C₁₆-C₁₈-PEG-MA/PETAE

| | | | |
|---|---|---|---|
| Initial charge: | 412 | g | butylacetate |
| Feed 1: | 37.5 | g | vinylpyrrolidone |
| | 6 | g | methacrylic acid |
| | 75.75 | g | vinylimidazole |
| | 30.0 | g | Plex ® 6877-O (25% strength |
| | | | C₁₆-C₁₈-PEG-MA in methyl |
| | | | methacrylate) |
| | 0.75 | g | pentaerythritol triallyl ether |
| Feed 2: | 38.2 | g | butyl acetate |
| | 0.15 | g | tert-butyl peroctoate |
| Feed 3: | 95.6 | g | butyl acetate |
| | 0.39 | g | tert-butyl peroctoate |
| Feed 4: | 100 | g | methyl chloride |

At 85 to 88° C., feed 1 and feed 2 were added over 2 hours to a pressurized apparatus fitted with stirrer, reflux condenser, internal thermometer and four separate feed devices. The reaction mixture was stirred for a further 2 h at about 88° C. Feed 3 was then metered in over 30 minutes and the mixture was after-polymerized at 90° C. for 3 hours. The product which precipitated out in the form of a white powder was quaternized with methyl chloride (feed 4) in about 1 h at about 90° C. The powder was removed using a suction filter, washed twice with acetone and dried under reduced pressure at 40° C.

The polymers 41 to 60 and 62 to 93 in table II below were prepared according to this procedure. In examples Nos. 74 to 93, Belsil® DMC 6031 was initially introduced in the initial charge.

TABLE II
VI/MAA/VP precipitation polymers

| | VP | VCap | MAA | VI | | SMA | MMA | C₁₆-C₁₈- | | | Belsil ® | Quaternization |
|---|---|---|---|---|---|---|---|---|---|---|---|---|
| Example | [% by | [% by | [% by | [% by | DMAEMA | [% by | [% by | PEG-MA | ODVE | PETAE | DMC 6031 | with |
| No. | wt.] | wt.] | wt.] | wt.] | [% by wt.] | wt.] | wt.] | [% by wt.] | [% by wt.] | [% by wt.] | [% by wt.] | with CH₃—Cl |
| 41 | 29.5 | — | 2 | 68 | — | — | — | — | — | 0.5 | — | ≧70% |
| 42 | 79.5 | — | 4 | 16 | — | — | — | — | — | 0.5 | — | ≧70% |
| 43 | 71 | — | 3.5 | 25 | — | — | — | — | — | 0.5 | — | ≧70% |
| 44 | 66.5 | — | 3 | 30 | — | — | — | — | — | 0.5 | — | ≧70% |
| 45 | 56.5 | — | 3 | 40 | — | — | — | — | — | 0.5 | — | ≧70% |
| 46 | 46.5 | — | 3 | 50 | — | — | — | — | — | 0.5 | — | ≧70% |
| 47 | 60 | 11.5 | 3 | 25 | — | — | — | — | — | 0.5 | — | ≧70% |
| 48 | 56 | 15 | 3.5 | 25 | — | — | — | — | — | 0.5 | — | ≧80% |
| 49 | 56.5 | 10 | 3 | 30 | — | — | — | — | — | 0.5 | — | ≧70% |
| 50 | 46 | 20 | 3.5 | 26 | — | — | — | — | — | 0.5 | — | ≧70% |
| 51 | 27 | — | 3 | 67 | — | 2.5 | — | — | — | 0.5 | — | ≧70% |
| 52 | 70 | — | 3 | 25 | — | 1.5 | — | — | — | 0.5 | — | ≧70% |
| 53 | 59 | — | 4 | 30 | | 1.5 | — | — | — | 0.5 | — | ≧70% |
| 54 | 58 | — | 4 | 35 | — | 2.5 | — | — | — | 0.5 | — | ≧70% |
| 55 | 79.6 | — | 3 | 10 | — | — | 7 | — | — | 0.4 | — | ≧80% |
| 56 | 25 | — | 3 | 62 | — | — | 9.5 | — | — | 0.5 | — | ≧70% |
| 57 | 38 | — | 3.5 | 43 | — | — | 15 | — | — | 0.5 | — | ≧70% |
| 58 | 61.5 | — | 4 | 25 | — | — | 10 | — | — | 0.5 | — | ≧70% |
| 59 | 46 | — | 3.5 | 30 | — | — | 20 | — | — | 0.5 | — | ≧70% |
| 60 | 25 | — | 3 | 61.5 | — | — | 7.5 | 2.5 | — | 0.5 | — | ≧70% |
| 61 | 25 | — | 4 | 50.5 | — | — | 15 | 5 | — | 0.5 | — | ≧70% |
| 62 | 46.7 | — | 4 | 34 | — | — | 7.5 | 2.5 | — | 0.3 | — | ≧70% |
| 63 | 56.5 | — | 4 | 29 | — | — | 7.5 | 2.5 | — | 0.5 | — | ≧70% |
| 64 | 61.5 | — | 3 | 25 | — | — | 7.5 | 2.5 | — | 0.5 | — | ≧70% |
| 65 | 66.5 | — | 3 | 25 | — | — | 3.75 | 1.25 | — | 0.5 | — | ≧70% |
| 66 | 72 | — | 3.5 | 20 | — | — | 3 | 1 | — | 0.5 | — | ≧80% |
| 67 | 79.6 | — | 3 | 12 | — | — | 3.75 | 1.25 | — | 0.5 | — | ≧80% |
| 68 | 74.5 | — | 4 | 16 | — | — | 3.75 | 1.25 | — | 0.5 | — | ≧70% |
| 69 | 55 | 10 | 3 | 30 | — | — | | — | 1.5 | 0.5 | — | ≧70% |
| 70 | 68 | — | 4 | 26 | — | — | — | — | 1.5 | 0.5 | — | ≧70% |
| 71 | 65 | — | 4 | 16 | 10 | 4.5 | — | — | — | 0.5 | — | ≧70% |
| 72 | 65 | — | 5 | 10 | 10 | — | 9.5 | — | — | 0.5 | — | ≧70% |
| 73 | 64.5 | — | 5 | 20 | 5 | — | 3.75 | 1.25 | — | 0.5 | — | ≧70% |
| 74 | 71 | — | 3.5 | 25 | — | — | — | — | — | 0.5 | 0.5 | ≧70% |
| 75 | 66.5 | — | 3 | 30 | — | — | — | — | — | 0.5 | 1 | ≧70% |
| 76 | 56.5 | — | 3 | 40 | — | — | — | — | — | 0.5 | 2 | ≧70% |
| 77 | 60 | 11.5 | 3 | 25 | — | — | — | — | — | 0.5 | 0.5 | ≧70% |
| 78 | 56 | 15 | 3.5 | 25 | — | — | — | — | — | 0.5 | 0.5 | ≧80% |
| 79 | 56.5 | 10 | 3 | 30 | — | — | — | — | — | 0.5 | 1 | ≧70% |
| 80 | 46 | 20 | 3.5 | 26 | — | — | — | — | — | 0.5 | 1 | ≧70% |
| 81 | 70 | — | 3 | 25 | — | 1.5 | — | — | — | 0.5 | 0.5 | ≧70% |
| 82 | 59 | — | 4 | 30 | | 1.5 | — | — | — | 0.5 | 0.5 | ≧70% |
| 83 | 58 | — | 4 | 35 | — | 2.5 | — | — | — | 0.5 | 0.5 | ≧70% |
| 84 | 61.5 | — | 4 | 25 | — | — | 10 | — | — | 0.5 | 1 | ≧70% |
| 85 | 46 | — | 3.5 | 30 | — | — | 20 | — | — | 0.5 | 1 | ≧70% |
| 86 | 46.7 | — | 4 | 34 | — | — | 7.5 | 2.5 | — | 0.3 | 1 | ≧70% |
| 87 | 56.5 | — | 4 | 29 | — | — | 7.5 | 2.5 | — | 0.5 | 1 | ≧70% |
| 88 | 66.5 | — | 3 | 25 | — | — | 3.75 | 1.25 | — | 0.5 | 1 | ≧70% |
| 89 | 72 | — | 3.5 | 20 | — | — | 3 | 1 | — | 0.5 | 0.5 | ≧80% |
| 90 | 55 | 10 | 3 | 30 | — | — | | — | 1.5 | 0.5 | 2 | ≧70% |
| 91 | 68 | — | 4 | 26 | — | — | — | — | 1.5 | 0.5 | 1 | ≧70% |
| 92 | 65 | — | 5 | 10 | 10 | — | 9.5 | — | — | 0.5 | 1 | ≧70% |
| 93 | 64.5 | — | 5 | 20 | 5 | — | 3.75 | 1.25 | — | 0.5 | 1 | ≧70% |

VP = N-vinylpyrrolidone
VCap = N-vinylcaprolactam
MAA = methacrylic acid
VI = N-vinylimidazole
DMAEMA = N,N-dimethylaminoethyl methacrylate
SMA = stearyl methacrylate
MMA = methyl methacrylate
C₁₆-C₁₈-PEG-MA = polyethylene glycol methacrylate terminated with a C₁₆-C₁₈-fatty alcohol mixture
ODVE octadecyl vinyl ether
PETAE pentaerythritol triallyl ether
MMA and C₁₆-C₁₈-PEG-MA can also be used together, e.g. in the form of the commercial product Plex ® 6877-O, Degussa, Germany.
Belsil ® DMC 6031: ethoxylated/propoxylated dimethylsiloxane, Wacker, Germany.

### EXAMPLE 114 (VARIANT B)

### Polymerization of VI/MAA/VP/PETAE in the Presence of an Ethoxylated Dimethylsiloxane Using Two Free Radical Initiators with Varying Decomposition Temperature

| | | | |
|---|---|---|---|
| Initial charge: | 613 | g | butyl acetate |
| | 2 | g | Belsil ® DMC 6031 |
| | 1 | g | Trigonox ® 101 (2,5-dimethyl-2,5- |
| | | | di(tert-butylperoxy)hexane) |
| Feed 1: | 94 | g | vinylpyrrolidone |
| | 102 | g | vinylimidazole |
| | 6.95 | g | methacrylic acid |
| | 1.2 | g | pentaerythritol triallyl ether |
| Feed 2: | 35 | g | n-butyl acetate |
| | 0.2 | g | tert-butyl peroctoate |
| Feed 3 | 175 | g | n-butyl acetate |
| | 1.0 | g | tert-butyl peroctoate |
| Feed 4: | 175 | g | n-butyl acetate |
| | 1.0 | g | tert-butyl peroctoate |

The initial charge was heated under a nitrogen atmosphere to 90° C. in an apparatus fitted with stirrer, reflux counter, internal thermometer and four feed devices. Feeds 1 and 2 were added over the course of 3 h and the mixture was stirred for a further 1.5 h at 90° C. Feed 3 was added at 100° C. in 1 h, and the reaction mixture was stirred for 1 h at this temperature. Feed 4 was then added at 100° C. over the course of 1 h and the mixture was again stirred for 2 h at 100° C. The temperature was increased to 125° C. and stirred for a further 2 h at this temperature. The white suspension obtained was then quaternized with 50 g of methyl chloride. The product was filtered, washed with acetone and dried under reduced pressure at 70° C.

All of the products of variant B as in table III were prepared analogously.

### EXAMPLE 96 (VARIANT C)

### Polymerization of AS/DMAPMAM/SMA/PETAE in the Presence of an Ethoxylated Dimethylsiloxane Using Two Free Radical Initiators with Varying Decomposition Temperature

| | | | |
|---|---|---|---|
| Initial charge: | 800 | g | ethyl acetate |
| | 1.6 | g | Belsil ® DMC 6031 |
| | 1 | g | tert-butyl peroctoate |
| Feed 1: | 164 | g | acrylic acid |
| Feed 2: | 7.8 | g | DMAPMAM |
| | 1.3 | g | pentaerythritol triallyl ether |
| | 3.5 | g | stearyl methacrylate |
| Feed 3 | 80 | g | ethyl acetate |
| | 0.4 | g | lauroyl peroxide |
| Feed 4: | 200 | g | ethyl acetate |
| | 0.4 | g | lauroyl peroxide |

The initial charge was heated to 75° C. under a nitrogen atmosphere and with stirring in an apparatus fitted with stirrer, reflux counter, internal thermometer and four feed devices. Feeds 1, 2 and 3 were added over the course of 3 h and the mixture was stirred for a further 2 h at 75° C. Feed 4 was added in 1 h at 80° C. and the mixture was then stirred for a further 1 h. The temperature was increased to 100° C. and, at this temperature, the mixture was stirred for a further 3 h. The resulting white suspension was then quaternized with 40 g of methyl chloride. The product was filtered, washed with acetone and dried under reduced pressure at 70° C.

All of the products of variant C as in table III were prepared analogously.

### EXAMPLE 94 (VARIANT D)

### Polymerization of AA/DMAPMAM/SMA/PETAE in the Presence of an Ethoxylated Dimethylsiloxane Using Two Free Radical Initiators with Varying Decomposition Temperature

| | | | |
|---|---|---|---|
| Initial charge: | 670 | g | ethyl acetate/cyclohexane (65:35) |
| | 2 | g | Belsil ® DMC 6031 |
| | 50 | g | feed 1 |
| | 14 | g | feed 2 |
| | 1.5 | g | pentaerythritol triallyl ether |
| | 1.5 | g | tert-butyl peroctoate |
| Feed 1: | 142.5 | g | acrylic acid |
| | 3 | g | stearyl methacrylate |
| | 3 | g | dimethylaminopropylmethacrylamide |
| | 100 | g | ethyl acetate/cyclohexane (65:35) |
| | 4.3 | g | anhydrous K₂CO₃ |
| Feed 2: | 70 | g | ethyl acetate/cyclohexane (65:35) |
| | 0.35 | g | Trigonox ® EHP-C75 (75% strength) |
| Feed 3 | 70 | g | ethyl acetate/cyclohexane (65:35) |
| | 1.0 | g | Trigonox ® EHP-C75 (75% strength) |

The initial charge was heated to 50° C. under a nitrogen atmosphere and with stirring in an apparatus fitted with stirrer, reflux counter, internal thermometer and three feed devices. Feed 1 was added over the course of 1.5 h and feed 2 was added over the course of 2 h and the mixture was stirred for a further 2 h at 60° C. Feed 3 was added in 1 h at 60° C. and the mixture was then stirred for a further 2 h at 70° C. The temperature was increased to 100° C. and, at this temperature, the mixture was stirred for a further 3 h. The resulting white suspension was filtered, washed with acetone and dried under reduced pressure at 70° C.

All of the products of variant D as in table III were prepared analogously.

TABLE III

| | (EO)- | | | | | | | | | Preparation |
|---|---|---|---|---|---|---|---|---|---|---|
| Ex. | Silicone^{#} | VP^{#} | MAA^{#} | AA^{#} | VI^{#} | DMAPMAM^{#} | SMA^{#} | EGDMA^{#} | PETAE^{#} | variant |
| 94 | 1 | | | 95 | | 2.0 | 2.0 | | 1.0 | D |
| 95 | 1 | | | 92 | | 2.0 | 5.0 | | 1.0 | D |
| 96 | 1 | | | 92 | | 5.0 | 2.0 | | 1.0 | C |
| 97 | 1 | | 90 | | | 8.5 | | 1.5 | | B |
| 98 | 1 | | 90 | | | 5.0 | 3.5 | 1.5 | | B |
| 99 | 1 | 23.8 | 70 | | | 5.0 | | 1.2 | | B |
| 100 | 1 | 45.8 | 50 | | | 3.0 | | 1.2 | | B |
| 101 | 1 | 45.8 | | 50 | | 3.0 | | | 1.2 | D |
| 102 | 1 | 45.0 | | 48 | | 3.0 | 2.8 | | 1.2 | D |
| 103 | | | | 95 | | 2.0 | 2.0 | | 1.0 | D |
| 104 | | | | 92 | | 2.0 | 5.0 | | 1.0 | D |
| 105 | | | | 92 | | 5.0 | 2.0 | | 1.0 | C |
| 106 | | | 90 | | | 8.5 | | 1.5 | | B |
| 107 | | | 90 | | | 5.0 | 3.5 | 1.5 | | B |
| 108 | | 23.8 | 70 | | | 5.0 | | 1.2 | | B |
| 109 | | 45.8 | 50 | | | 3.0 | | 1.2 | | B |
| 110 | | 45.8 | | 50 | | 3.0 | | | 1.2 | D |
| 111 | | 45.0 | | 48 | | 3.0 | 2.8 | | 1.2 | D |
| 112 | 1 | 70.0 | 3.4 | | 26 | | | | 0.6 | B* |
| 113 | 1 | 60.0 | 3.4 | | 36 | | | | 0.6 | B |
| 114 | 1 | 46.0 | 3.4 | | 50 | | | | 0.6 | B |
| 115 | 1 | 16.0 | 3.4 | | 80 | | | | 0.6 | B |
| 116 | 1 | | 3.4 | | 95 | | | | 0.6 | B |
| 117 | 1 | | 3.0 | | 94 | | | | 0.5 | B |
| 118 | | 70.0 | 3.4 | | 26 | | | | 0.6 | B* |
| 119 | | 60.0 | 3.4 | | 36 | | | | 0.6 | B |
| 120 | | 46.0 | 3.4 | | 50 | | | | 0.6 | B |
| 121 | | 16.0 | 3.43 | | 80 | | | | 0.6 | B |
| 122 | | | 3.4 | | 95 | | | | 0.6 | B |
| 123 | | | 3.0 | | 94 | | | | 0.5 | B |

Ex. Example
^{#}The quantitative data are in % by weight, based on the unsaturated compounds used for the polymerization, the parts by weight of Si-containing compound are given separately
(EO)-silicone Belsil ® 6031, ethoxylated dimethylsiloxane
VP vinylpyrrolidone
MAA methacrylic acid
AA acrylic acid
VI vinylimidazole
DMAPMAM dimethylaminopropylmethacrylamide
SMA stearyl methacrylate
EGDMA ethylene glycol dimethacrylate
PETAE pentaerythritol triallyl ether
EMA ethyl methacrylate
n-BA n-butyl acrylate
PLEX-O Plex ® 6877-0 = methacrylic acid ester of a C₁₆-C₁₈-fatty alcohol alkoxylated with 25 mol of ethylene oxide as 25% strength solution in methyl methacrylate
*quaternized to 70% with CH₃Cl

### II. APPLICATION EXAMPLES

### Application Examples 1-58

Hair gels containing a nonionic hair-setting agent:

| CTFA | % by wt. |
|---|---|
| Phase 1: | |
| Polymer (anionically ampholytic) from Example No. | 2.0 |
| 1/2/3/4/5/6/7/8/9/10/11/12/13/14/15/16/17/ | |
| 18/19/20/21/22/23/24/25/26/27/28/29/30/31/ | |
| 32/33/34/35/36/37/38/39/40/94/95/96/97/98/ | |
| 99/100/101/102/103/104/105/106/107/108/109/ | |
| 110/111 | |
| Luviskol ® K90 Polyvinylpyrrolidone | 1.5 |
| Water, dist. | 46.5 |

Further additive: preservative, e.g. Euxyl ® K100, perfume, etc.
with triethanolamine (50% strength solution) adjust to pH between 6.7 and
7.2

| | |
|---|---|
| Phase 2: | |
| Water, dist. | ad 100 |

Preparation:

Phase 1 was weighed and homogenized with stirring at a temperature in the range from 20 to 50° C. After about 3 hours, a milky dispersion had formed. Triethanolamine was added with stirring. After about 2 hours, a (virtually) homogeneous, high-viscosity gel had formed. Phase 2 was then stirred slowly into phase 1. The gel was stirred at room temperature for a further hour. This produced a stable, clear gel.

In the same way, a standard gel (comparison gel CG) containing Carbopol® 940 and Luviskol® K90 was prepared, with phase 1 comprising 0.5% by weight of Carbopol® 940 and 3% by weight of Luviskol® K90 instead of 2.0% by weight of the polymer from Example No. 1 and 1.5% by weight of Luviskol K90.

Hair gels of Application Examples 5, 16, 30 and 35 which comprised the polymer from Example Nos. 5, 16, 30 and 35, respectively, in each case in an amount of 2.0% by weight, and also Luviskol® K90, in each case in an amount of 1.5% by weight, were investigated with regard to their application properties (clarity, stickiness, wet combability and setting). The comparison used was the comparison gel CG described above. The polymer content both of the gels according to the invention and also of the comparison gel was thus 3.5%.

The test was based on the following evaluation scale:

| | Clarity | Stickiness | Wet combability | Setting |
|---|---|---|---|---|
| Grade 1 | clear | not sticky | very good | very good |
| Grade 2 | slightly cloudy | slightly sticky | good | good |
| Grade 3 | cloudy | sticky | average | just good |

| | | | Wet | |
|---|---|---|---|---|
| | Clarity | Stickiness | combability | Setting |
| Comparison gel, CG | 1-2 | 2-3 | 2-3 | 1-2 |
| Gel containing polymer | 1 | 2 | 1-2 | 1 |
| from Ex. No. 5 | | | | |
| Gel containing polymer | 1 | 1-2 | 2 | 1 |
| from Ex. No. 16 | | | | |
| Gel containing polymer | 1 | 1-2 | 1-2 | 1 |
| from Ex. No. 30 | | | | |
| Gel containing polymer | 1-2 | 1-2 | 2 | 1 |
| from Ex. No. 35 | | | | |

### Application Examples 59-123

Hair gels containing a nonionic hair-setting agent:

| CTFA | % by wt. |
|---|---|
| Phase 1: | |
| Polymer (cationically ampholytic) from Example No. | 2.0 |
| 41/42/43/44/45/46/47/48/49/50/ | |
| 51/52/53/54/55/56/57/58/59/60/61/62/63/ | |
| 64/65/66/67/68/69/70/71/72/73/74/75/76/ | |
| 77/78/79/80/81/82/83/84/85/86/87/88/89/ | |
| 90/91/92/93/112/113/114/115/116/117/118/ | |
| 119/120/121/123 | |
| Luviskol ® K90 Polyvinylpyrrolidone | 1.5 |
| Water, dist. | 46.5 |
| Phase 2: | |
| Water, dist. | 100.0 |

Further additive: preservative, e.g. Euxyl ® K100, perfume, etc.

Preparation:

Phase 1 was weighed in and homogenized with stirring at a temperature in the range from 20 to 50° C. After about 2 hours, a (virtually) homogeneous, solid gel had formed. Phase 2 was then stirred slowly into phase 1. The gel was stirred at room temperature for another hour. This produced a stable, clear gel.

In the same way, a standard gel (comparison gel CG) was prepared with Carbopol® 940 and Luviskol® K90, where phase 1 comprised 0.5% by weight of Carbopol® 940 and 3% by weight of Luviskol® K90 instead of 2.0% by weight of the polymer from Example No. 1 and 1.5% by weight of Luviskol® K90.

Hair gels of Application Examples 50, 51, 58, 80, 81 and 86, which comprised the polymer from Example Nos. 50, 51, 58, 80, 81 and 86, respectively, in each case in an amount of 2.0% by weight, and also Luviskol® K90, in each case in an amount of 1.5% by weight, were investigated with regard to their application properties (clarity, stickiness, wet combability and setting). The comparison used was the comparison gel CG described above. The polymer content of both the gels according to the invention and also of the comparison gel was thus 3.5%.

The test was based on the above evaluation scale.

Test Results:

| | | | Wet | |
|---|---|---|---|---|
| | Clarity | Stickiness | combability | Setting |
| Comparison gel CG | 1-2 | 2-3 | 2-3 | 1-2 |
| Gel containing polymer | 1 | 1-2 | 1-2 | 1 |
| from Ex. No. 50 | | | | |
| Gel containing polymer | 1-2 | 1-2 | 1-2 | 1 |
| from Ex. No. 51 | | | | |
| Gel containing polymer | 1 | 1-2 | 1-2 | 1 |
| from Ex. No. 58 | | | | |
| Gel containing polymer | 1 | 1-2 | 1-2 | 1 |
| from Ex. No. 80 | | | | |
| Gel containing polymer | 1 | 1-2 | 1-2 | 1 |
| from Ex. No. 81 | | | | |
| Gel containing polymer | 1 | 1-2 | 1-2 | 1-2 |
| from Ex. No. 86 | | | | |

Ex. Example

### Application Examples 124-181

Hair gels containing a nonionic hair-setting agent:

| CTFA | % by wt. |
|---|---|
| Phase 1: | |
| Polymer (anionically ampholytic) from Example No. | 1.8 |
| 1/2/3/4/5/6/7/8/9/10/11/12/13/14/15/16/17/ | |
| 18/19/20/21/22/23/24/25/26/27/28/29/30/31/ | |
| 32/33/34/35/36/37/38/39/40/94/95/96/97/98/ | |
| 99/100/101/102/103/104/105/106/107/108/109/ | |
| 110/111 | |
| Luviskol ® K90 Polyvinylpyrrolidone | 1.0 |
| Water, dist | 46.8 |

Further additive: preservative, e.g. Euxyl ® K100, perfume, etc.
with triethanolamine (50% strength solution) adjust to pH between 6.7 and
7.2

| | |
|---|---|
| Phase 2: | |
| Water, dist. | ad 100 |

Preparation:

Phase 1 was weighed in and homogenized with stirring at a temperature in the range from 20 to 50° C. After about 3 hours, a milky dispersion had formed. Triethanolamine was added with stirring. After about 2 hours, a (virtually) homogeneous, high-viscosity gel had formed. Phase 2 was then stirred slowly into phase 1. The gel was stirred at room temperature for another hour. This produced a stable, clear gel.

Hair gels of Application Examples 128, 139, 153 and 158, which comprise the polymer from Example Nos. 5, 16, 30 and 35, respectively, in each case in an amount of 1.8% by weight, and also Luviskol® K90, in each case in an amount of 1.0% by weight, were investigated with regard to their application properties. The comparison gel used was that described above. The polymer content of the gels according to the invention was thus only 2.8%, that of the comparison gel was 3.5%. The test was based on the above evaluation scale:

| | | | Wet | |
|---|---|---|---|---|
| | Clarity | Stickiness | combability | Setting |
| Comparison gel, CG | 1-2 | 2-3 | 2-3 | 1-2 |
| Gel containing polymer | 1 | 2 | 2 | 1-2 |
| from Ex. No. 5 | | | | |
| Gel containing polymer | 1 | 1-2 | 2 | 1-2 |
| from Ex. No. 16 | | | | |
| Gel containing polymer | 1 | 1-2 | 1-2 | 1-2 |
| from Ex. No. 30 | | | | |
| Gel containing polymer | 1-2 | 1-2 | 2 | 1-2 |
| from Ex. No. 35 | | | | |

As the table shows, with the gels according to the invention better formulations are obtained even at a reduced polymer content than with the comparison gel.

### Application Examples 182-246

Hair gels containing a nonionic hair-setting agent:

| CTFA | % by wt. |
|---|---|
| Phase 1: | |
| Polymer (cationically ampholytic) from Example No. | 2.0 |
| 41/42/43/44/45/46/47/48/49/50/51/52/53/54/ | |
| 55/56/57/58/59/60/61/62/63/64/65/66/67/68/ | |
| 69/70/71/72/73/74/75/76/77/78/79/80/81/82/ | |
| 83/84/85/86/87/88/89/90/91/92/53/112/113/ | |
| 114/115/116/117/118/119/120/121/122/123 | |
| Luviskol ® K90 Polyvinylpyrrolidone | 0.7 |
| Water, dist. | 46.8 |

Further additive: preservative, e.g. Euxyl ® K100, perfume, etc.
with triethanolamine (50% strength solution) adjust to pH 6.7-7.2.

| | |
|---|---|
| Phase 2: | |
| Water dist. | ad 100.0 |

Preparation:

Phase 1 was weighed in and homogenized with stirring at a temperature in the range from 20 to 50° C. After about 2 hours, a (virtually) homogeneous, solid gel had formed. Phase 2 was then stirred slowly into phase 1. The gel was stirred at room temperature for another hour. This produced a stable, clear gel.

Hair gels of application examples 191, 192, 199, 221, 222 and 227, which comprise the polymer from Example Nos. 50, 51, 58, 80, 81 and 86, respectively, in each case in an amount of 2.0% by weight, and also Luviskol® K90, in each case in an amount of 0.7% by weight, were investigated with regard to their application properties. The comparison gel used was that described above. The polymer content of the gels according to the invention was thus only 2.7%, that of the comparison gel was 3.5%. The test was based on the above evaluation scale:

Test Results:

| | | | Wet | |
|---|---|---|---|---|
| | Clarity | Stickiness | combability | Setting |
| Comparison gel CG | 1-2 | 2-3 | 2-3 | 1-2 |
| Gel containing polymer | 1 | 1-2 | 1-2 | 1 |
| from Ex. No. 50 | | | | |
| Gel containing polymer | 1-2 | 1-2 | 1-2 | 1-2 |
| from Ex. No. 51 | | | | |
| Gel containing polymer | 1 | 1-2 | 1-2 | 1 |
| from Ex. No. 58 | | | | |
| Gel containing polymer | 1 | 1-2 | 1-2 | 1 |
| from Ex. No. 80 | | | | |
| Gel containing polymer | 1-2 | 1-2 | 1-2 | 1 |
| from Ex. No. 81 | | | | |
| Gel containing polymer | 1-2 | 1-2 | 1-2 | 1-2 |
| from Ex. No. 86 | | | | |

As the table shows, with the gels according to the invention, better formulations are achieved, even at reduced polymer content, than with the comparison gel.

### Application Examples 247-304

Hair gels containing poly(vinylpyrrolidone/vinyl acetate)

| CTFA | % by wt. |
|---|---|
| Phase 1: | |
| Polymer (anionically ampholytic) from Example No. | 2.0 |
| 1/2/3/4/5/6/7/8/9/10/11/12/13/14/15/16/17/ | |
| 18/19/20/21/22/23/24/25/26/27/28/29/30/31/ | |
| 32/33/34/35/36/37/38/39/40/94/95/96/97/98/ | |
| 99/100/101/102/103/104/105/106/107/108/109/ | |
| 110/111 | |
| Luviskol ® VA64 Poly(vinylpyrrolidone/ | 1.5 |
| vinyl acetate) | |
| Water, dist. | 46.5 |

Further additive: preservative, e.g. Euxyl ® K100, perfume etc.
with triethanolamine (50% strength solution) adjust to pH between 6.7-7.2.

| | |
|---|---|
| Phase 2: | |
| Water, dist. | ad 100 |

Preparation:

Phase 1 was weighed in and homogenized with stirring at a temperature in the range from 20 to 50° C. After about 3 hours, a milky dispersion had formed. Triethanolamine was added with stirring. After about 2 hours, a (virtually) homogeneous, high-viscosity gel had formed. Phase 2 was then stirred slowly into phase 1. The gel was stirred at room temperature for a further hour. This produced a stable, virtually clear to clear gel.

### Application Examples 305-369

Hair gels containing poly(vinylpyrrolidone/vinyl actate):

| CTFA | % by wt. |
|---|---|
| Phase 1: | |
| Polymer (cationically ampholytic) from Example No. | 2.0 |
| 41/42/43/44/45/46/47/48/49/50/51/52/53/54/ | |
| 55/56/57/58/59/60/61/62/63/64/65/66/67/68/ | |
| 69/70/71/72/73/74/75/76/77/78/79/80/81/82/ | |
| 83/84/85/86/87/88/89/90/91/92/53/112/113/ | |
| 114/115/116/117/118/119/120/121/122/123 | |
| Luviskol ® VA64 Poly(vinylpyrrolidone/ | 1.5 |
| vinyl acetate) | |
| Water, dist. | 46.5 |
| Phase 2: | |
| Water, dist. | ad 100 |

Further additive: preservative, e.g. Euxyl ® K100, perfume, etc.

Preparation:

Phase 1 was weighed in and homogenized with stirring at a temperature in the range from 20 to 50° C. After about 2 hours, a (virtually) homogeneous, solid gel had formed. Phase 2 was then stirred slowly into phase 1. The gel was stirred at room temperature for a further hour. This produced a stable, virtually clear to clear gel.

### Application Examples 370-427

Hair gels containing a cationic hair polymer

| CTFA | % by wt. |
|---|---|
| Phase 1: | |
| Polymer (anionically ampholytic) from Example No. | 2.2 |
| 1/2/3/4/5/6/7/8/9/10/11/12/13/14/15/16/17/ | |
| 18/19/20/21/22/23/24/25/26/27/28/29/30/31/ | |
| 32/33/34/35/36/37/38/39/40/94/95/96/97/98/ | |
| 99/100/101/102/103/104/105/106/107/108/109/ | |
| 110/111 | |
| Luviskol ® K90 Polyvinylpyrrolidone | 0.8 |
| Luviquat ® Supreme (BASF AG) Polyquaternium-68 | 0.5 |
| Water, dist. | 46.5 |

Further additive: preservative, e.g. Euxyl ® K100, perfume, etc.
with triethanolamine (50% strength solution) adjust to pH between 6.7-7.2

| | |
|---|---|
| Phase 2: | |
| Water, dist. | ad 100 |

Preparation:

Phase 1 was weighed in and homogenized with stirring at a temperature in the range from 20 to 50° C. After about 3 hours, a milky dispersion had formed. Triethanolamine was added with stirring. After about 2 hours, a (virtually) homogeneous, high-viscosity gel had formed. Phase 2 was then stirred slowly into phase 1. The gel was stirred for a further 1 hour at room temperature. This produced a stable, virtually clear to clear gel.

### Application Examples 428-492

Hair gels containing a cationic hair polymer

| CTFA | % by wt. |
|---|---|
| Phase 1: | |
| Polymer (cationically ampholytic) from Example No. | 2.2 |
| 41/42/43/44/45/46/47/48/49/50/51/52/53/54/ | |
| 55/56/57/58/59/60/61/62/63/64/65/66/67/68/ | |
| 69/70/71/72/73/74/75/76/77/78/79/80/81/82/ | |
| 83/84/85/86/87/88/89/90/91/92/53/112/113/ | |
| 114/115/116/117/118/119/120/121/122/123 | |
| Luviskol ® K90 Polyvinylpyrrolidone | 0.8 |
| Luviset ® Clear (BASF) Poly(VP/methacrylamide/ | 0.5 |
| VI) | |
| Water, dist. | 46.5 |
| Phase 2: | |
| Water dist. | ad 100.0 |

Further additive: preservative, e.g. Euxyl ® K100, perfume, etc.

Preparation:

Phase 1 was weighed in and homogenized with stirring at a temperature in the range from 20 to 50° C. After about 2 hours, a (virtually) homogeneous, solid gel had formed. Phase 2 was then stirred slowly into phase 1. The gel was stirred for a further hour at room temperature. This produced a stable, virtually clear to clear gel.

### Application Examples 493-550

Flexible hair-setting gels

| CTFA | % by wt. |
|---|---|
| Phase 1: | |
| Polymer (anionically ampholytic) from Example No. | 2.0 |
| 1/2/3/4/5/6/7/8/9/10/11/12/13/14/15/16/17/ | |
| 18/19/20/21/22/23/24/25/26/27/28/29/30/31/ | |
| 32/33/34/35/36/37/38/39/40/94/95/96/97/98/ | |
| 99/100/101/102/103/104/105/106/107/108/109/ | |
| 110/111 | |
| Luviskol ® K90 Polyvinylpyrrolidone | 1.0 |
| D-Panthenol, USP Panthenol | 0.2 |
| Glycerol | 0.1 |
| Water, dist. | 46.7 |

Further additive: preservative, e.g. Euxyl ® K100, perfume, etc.
with triethanolamine (50% strength solution) adjust to pH between 6.7-7.2

| | |
|---|---|
| Phase 2: | |
| Water, dist. | ad 100 |

Preparation:

Phase 1 was weighed in and homogenized with stirring at a temperature in the range from 20 to 50° C. After about 3 hours, a milky dispersion had formed. Triethanolamine was added with stirring. After about 2 hours, a (virtually) homogeneous, high-viscosity gel had formed. Phase 2 was then stirred slowly into phase 1. The gel was stirred for a further 1 hour at room temperature. This produced a stable, virtually clear to clear gel.

### Application Examples 551-615

Flexible hair-setting gels

| CTFA | % by wt. |
|---|---|
| Phase 1: | |
| Polymer (cationically ampholytic) from Example No. | 2.0 |
| 41/42/43/44/45/46/47/48/49/50/51/52/53/54/ | |
| 55/56/57/58/59/60/61/62/63/64/65/66/67/68/ | |
| 69/70/71/72/73/74/75/76/77/78/79/80/81/82/ | |
| 83/84/85/86/87/88/89/90/91/92/53/112/113/ | |
| 114/115/116/117/118/119/120/121/122/123 | |
| Luviskol ® K90 Polyvinylpyrrolidone | 1.0 |
| D-Panthenol, USP Panthenol | 0.2 |
| Glycerol | 0.1 |
| Water, dist. | 46.7 |
| Phase 2: | |
| Water, dist. | ad 100.0 |

Further additive: preservative, e.g. Euxyl ® K100, perfume, etc.

Preparation:

Phase 1 was weighed in and homogenized with stirring at a temperature in the range from 20 to 50° C. After about 2 hours, a (virtually) homogeneous, solid gel had formed. Phase 2 was then stirred slowly into phase 1. The gel was stirred for a further 1 h at room temperature. This produced a stable, virtually clear to clear gel.

### Application Examples 616-673

Setting Foams

| CTFA | % by wt. |
|---|---|
| Phase 1: | |
| Polymer (anionically ampholytic) from Example No. | 0.5 |
| 1/2/3/4/5/6/7/8/9/10/11/12/13/14/15/16/17/ | |
| 18/19/20/21/22/23/24/25/26/27/28/29/30/31/ | |
| 32/33/34/35/36/37/38/39/40/94/95/96/97/98/ | |
| 99/100/101/102/103/104/105/106/107/108/109/ | |
| 110/111 | |
| Water | 28.0 |

with triethanolamine (50% strength) adjust to pH 6.8

| | |
|---|---|
| Phase 2: | |
| Luviset ® Clear | 1.2 |
| Water | 60.0 |
| Cremophor ® A 25 Ceteareth 25/BASF | 0.2 |
| Comperlan ® KD Coamide DEA/Henkel | 0.1 |

Further additive: perfume, preservative, etc.

| | |
|---|---|
| Dimethyl ether | 10.0 |

Preparation:

Phase 1: Stir polymer into water. With stirring, the polymer was neutralized with triethanolamine and dissolved. Slowly stir phase 2 into phase 1. Bottle and add propellant gas.

### Application Examples 674-738

Setting Foams

| CTFA | % by wt. |
|---|---|
| Phase 1: | |
| Polymer (cationically ampholytic) from Example No. | 0.5 |
| 41/42/43/44/45/46/47/48/49/50/51/52/53/54/ | |
| 55/56/57/58/59/60/61/62/63/64/65/66/67/68/ | |
| 69/70/71/72/73/74/75/76/77/78/79/80/81/82/ | |
| 83/84/85/86/87/88/89/90/91/92/53/112/113/ | |
| 114/115/116/117/118/119/120/121/122/123 | |
| Water | 28.0 |
| Phase 2: | |
| Luviquat ® Supreme (BASF) (VP/MAM/VI/QVI) | 1.2 |
| Water | 60.0 |
| Cremophor ® A 25 Ceteareth 25 | 0.2 |
| (BASF) | |
| Comperlan ® KD Coamide DEA | 0.1 |
| (Henkel) | |

Further additive: perfume, preservative, etc..

| | |
|---|---|
| Dimethyl ether | 10.0 |

Preparation:

Phase 1: Stir polymer into water. The mixture was then neutralized with triethanolamine with stirring and dissolved. Phase 2 was then stirred slowly into phase 1. The setting foam was then bottled and propellant gas was added.

### Application Examples 739-796

Setting Foams

| CTFA | % by wt. |
|---|---|
| Phase 1: | |
| Polymer (anionically ampholytic) from Example No. | 0.8 |
| 1/2/3/4/5/6/7/8/9/10/11/12/13/14/15/16/17/ | |
| 18/19/20/21/22/23/24/25/26/27/28/29/30/31/ | |
| 32/33/34/35/36/37/38/39/40/94/95/96/97/98/ | |
| 99/100/101/102/103/104/105/106/107/108/109/ | |
| 110/111 | |
| Water | 37.7 |

with triethanolamine (50% strength) adjust to pH 6.8

| | |
|---|---|
| Phase 2: | |
| Luviskol ® VA 64 | 1.2 |
| Cremophor ® A 25 Ceteareth 25/BASF | 0.2 |
| Comperlan ® KD Coamide DEA/Henkel | 0.1 |
| Water | 50.0 |

Further additive: perfume, preservative, etc.

| | |
|---|---|
| Dimethyl ether | 10.0 |

Preparation:

Phase 1: Stir polymer into water. With stirring, the polymer was neutralized with triethanolamine and dissolved. Slowly stir phase 2 into phase 1. Bottle and add propellant gas.

### Application Examples 797-861

Setting Foams

| CTFA | % by wt. |
|---|---|
| Phase 1: | |
| Polymer (cationically ampholytic) from Example No. | 0.7 |
| 41/42/43/44/45/46/47/48/49/50/51/52/53/54/ | |
| 55/56/57/58/59/60/61/62/63/64/65/66/67/68/ | |
| 69/70/71/72/73/74/75/76/77/78/79/80/81/82/ | |
| 83/84/85/86/87/88/89/90/91/92/53/112/113/ | |
| 114/115/116/117/118/119/120/121/122/123 | |
| Water | 37.7 |
| Phase 2: | |
| Luviskol ® VA 64 Poly(vinylpyrolidone/ | 1.0 |
| vinyl acetate) | |
| Luviskol ® K12 | 0.3 |
| Cremophor ® A 25 Ceteareth 25 | 0.2 |
| Comperlan ® KD Coamide DEA | 0.1 |
| Water | 50.0 |
| Further additive: perfume, preservative, etc. | |
| Dimethyl ether | 10.0 |

Preparation:

Phase 1: Stir polymer into water. The polymer was neutralized with triethanolamine with stirring and dissolved. Phase 2 was then slowly stirred into phase 1. Finally, the mixture was bottled and propellant gas was added.

### Application Examples 862-919

Shampoo (Without the Addition of Salt)

| CTFA | % by wt. |
|---|---|
| Phase 1: | |
| Polymer (anionically ampholytic) from Example No. | 1.0 |
| 1/2/3/4/5/6/7/8/9/10/11/12/13/14/15/16/17/ | |
| 18/19/20/21/22/23/24/25/26/27/28/29/30/31/ | |
| 32/33/34/35/36/37/38/39/40/94/95/96/97/98/ | |
| 99/100/101/102/103/104/105/106/107/108/109/ | |
| 110/111 | |
| Water | 48.0 |
| with triethanolamine (50% strength) adjust to pH 6.8 | |
| Phase 2: | |
| Texapon ® NSO 28% strength Sodium Laureth Sulfate/ | 50.0 |
| Henkel | |
| Comperlan ® KD Coamide DEA/Henkel | 1.0 |

Further additive: perfume, preservative, etc.

Preparation:

Weigh in and, with stirring, dissolve phases 1 and 2 separately and mix. Slowly stir phase 2 into phase 1.

### Application Examples 920-984

Shampoo

| CTFA | % by wt. |
|---|---|
| Phase 1: | |
| Polymer (cationically ampholytic) from Example No. | 1.0 |
| 41/42/43/44/45/46/47/48/49/50/51/52/53/54/ | |
| 55/56/57/58/59/60/61/62/63/64/65/66/67/68/ | |
| 69/70/71/72/73/74/75/76/77/78/79/80/81/82/ | |
| 83/84/85/86/87/88/89/90/91/92/53/112/113/ | |
| 114/115/116/117/118/119/120/121/122/123 | |
| Polyvinylpyrrolidone K30 | 0.5 |
| Water | 47.5 |
| Phase 2: | |
| Texapon ® NSO 28% strength Sodium Laureth | 50.0 |
| Sulphate (Henkel) | |
| Comperlan ® KD Coamide DEA | 1.0 |

Further additive: q.s. perfume oil, preservative

Preparation:

Weigh in and dissolve phases 1 and 2 separately with stirring, and mix. Slowly stir phase 2 into phase 1.

1. A method for modifying the rheology of a hair cosmetic composition, wherein an ampholytic copolymer which has a molar excess of anionogenic/anionic groups compared with cationogenic/cationic groups or which has a molar excess of cationogenic/cationic groups compared with anionogenic/anionic groups and which is obtainable by free-radical copolymerization of
a1) at least one compound with a free-radically polymerizable, α,β-ethylenically unsaturated double bond and at least one anionogenic and/or anionic group per molecule,
a2) at least one compound with a free-radically polymerizable, α,β-ethylenically unsaturated double bond and at least one cationogenic and/or cationic group per molecule,
b) at least one free-radically polymerizable crosslinking compound which comprises at least two α,β-ethylenically unsaturated double bonds per molecule,
c) if appropriate in the presence of at least one silicone compound comprising a polyether group and/or a free-radically polymerizable olefinically unsaturated double bond,
is used as rheology modifier.

2. The method according to claim 1, where the ampholytic copolymer is obtainable by free-radical copolymerization in accordance with the method of precipitation polymerization.

3. The method according to claim 1, where at least some of the compounds a1) and a2) are used in the form of a monomer pair, where the molar ratio of anionogenic groups of component a1) to cationogenic groups of component a2) is about 1:1.

4. The method according to claim 3, where the fraction in the monomer pair of the compounds used for the polymerization is at least 1% by weight.

5. The method according to claim 3, where the component a1) or a2) used in deficit is used completely as component of the monomer pair.

6. The method according to claim 1, where the ampholytic copolymer has a molar excess of anionogenic/anionic groups compared with cationogenic/cationic groups or a molar excess of cationogenic/cationic groups compared with anionogenic/anionic groups of at least 1.2:1.

7. The method according to claim 1, where the component a1) is chosen from acrylic acid, methacrylic acid and mixtures which comprise acrylic acid and/or methacrylic acid.

8. The method according to claim 1, where the component a2) is chosen from N-vinylimidazole compounds and mixtures which comprise at least one N-vinylimidazole compound.

9. The method according to claim 1, where the component a2) is chosen from N-[3-(dimethylamino)propyl]acrylamide, N-[3-(dimethylamino)propyl]methacrylamide, N,N-dimethylaminoethyl acrylate, N,N-dimethylaminoethyl methacrylate and mixtures thereof.

10. The method according to claim 1, wherein an ampholytic copolymer whose cationogenic groups have been quaternized following the copolymerization with CH₃Cl or dimethyl sulfate, is used.

11. The method according to claim 1, wherein an ampholytic copolymer whose anionogenic groups have been partially neutralized after the copolymerization with a base, preferably a trialkanolamine, specifically triethanolamine, is used.

12. The method according to claim 1, where the ampholytic copolymer additionally comprises at least one further monomer d) in copolymerized form which is chosen from α,β-ethylenically unsaturated amide-group-containing compounds of the general formula I 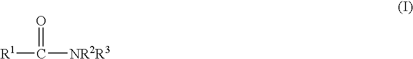
where
one of the radicals R¹ to R³ is a group of the formula CH₂═CR⁴— where R⁴═H or C₁-C₄-alkyl and the other radicals R¹ to R³, independently of one another, are H, alkyl, cycloalkyl, heterocycloalkyl, aryl or hetaryl,
where R¹ and R², together with the amide group to which they are bonded, may also be a lactam having 5 to 8 ring atoms,
where R² and R³, together with the nitrogen atom to which they are bonded, may also be a five- to seven-membered heterocycle,
with the proviso that the sum of the carbon atoms of the radicals R¹, R² and R³ is at most 8.

13. The method according to claim 1, where the ampholytic copolymer additionally comprises at least one further monomer e) in copolymerized form which is chosen from compounds of the general formulae III a), III b), III c), III d) and III e) 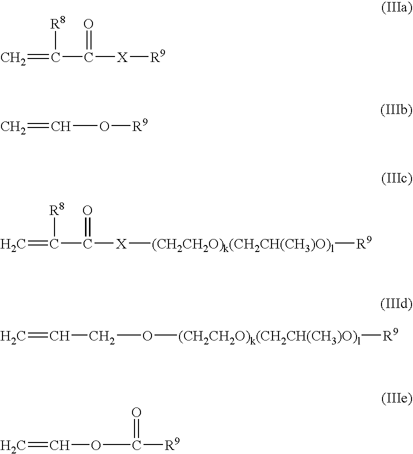
in which
the order of the alkylene oxide units is arbitrary,
k and l, independently of one another, are an integer from 0 to 1000, where the sum of k and l is at least 5,
R⁸ is hydrogen or C₁-C₄-alkyl,
R⁹ is C₈-C₃₀-alkyl or C₈-C₃₀-alkenyl, and
X is O or a group of the formula NR¹⁰, in which R¹⁰ is H, alkyl, alkenyl, cycloalkyl, heterocycloalkyl, aryl or hetaryl.

14. The method according to claim 1, where the ampholytic copolymer additionally comprises at least one compound f) in copolymerized form which is chosen from compounds of the general formulae III a*), III b*), III c*), III d*) and III e*) 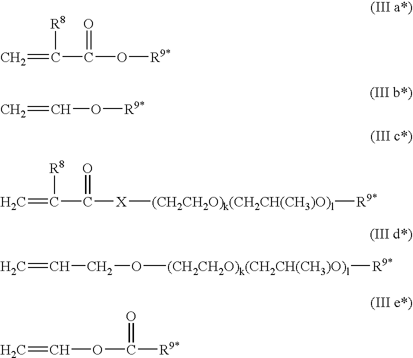
in which
the order of the alkylene oxide units is arbitrary,
k and l, independently of one another, are an integer from 0 to 1000, where the sum of k and l is at least 5,
R⁸ is hydrogen or C₁-C₄-alkyl,
R⁹* is hydrogen, C₁-C₈-alkyl or C₃-C₈-alkenyl, and
X is O or a group of the formula NR¹⁰, in which R¹⁰ is H, alkyl, alkenyl, cycloalkyl, heterocycloalkyl, aryl or hetaryl.

15. The method according to claim 1, where the ampholytic copolymer additionally comprises at least one compound g) in copolymerized form which is chosen from esters of α,β-ethylenically unsaturated mono- and dicarboxylic acids with C₂-C₃₀-diols, amides of α,β-ethylenically unsaturated mono- and dicarboxylic acids with C₂-C₃₀-amino alcohols which have a primary or secondary amino group, esters of allyl alcohol with C₁-C₇-monocarboxylic acids, polyether acrylates different from III c) and III c*), vinylaromatics, vinyl halides, vinylidene halides, C₂-C₈-monoolefins, nonaromatic hydrocarbons having at least two conjugated double bonds and mixtures thereof.

16. The method according to claim 1, wherein an ampholytic copolymer is used which is obtainable by free-radical copolymerization of
1 to 99% by weight of at least one compound a1) with a free-radically polymerizable, α,β-ethylenically unsaturated double bond and at least one anionogenic and/or anionic group per molecule,
2 to 96% by weight of the at least one compound a2)
0.05 to 5% by weight of at least one crosslinker b),
0 to 30% by weight, preferably 0 to 15% by weight, of at least one silicone compound c),
0 to 95% by weight of at least one amide-group-containing monomer d), preferably vinylpyrrolidone and/or vinylcaprolactam,
0 to 40% by weight of at least one compound e) which is preferably chosen from C₈-C₂₂-(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,
0 to 40% by weight of at least one monomer f) which is preferably chosen from C₁-C₆-(meth)acrylates, in particular methyl(meth)acrylate, ethyl(meth)acrylate, n-butyl(meth)acrylate and mixtures thereof.

17. A copolymer A) according to claim 1 which has a molar excess of anionogenic/anionic groups compared with cationogenic/cationic groups of greater than 1:1.

18. The copolymer A) according to claim 17, where up to 60% by weight of component a1), based on the total weight of the monomers a1), are replaced by at least one monomer d.

19. The method according to claim 1, where an ampholytic copolymer is used that is obtainable by free-radical copolymerization of
at least 2% by weight, based on the total weight of the monomers used for the polymerization, of at least one monomer pair from N-vinylimidazole and acrylic acid and/or methacrylic acid,
5 to 70% by weight of methacrylic acid and/or acrylic acid,
0.1 to 2% by weight of at least one crosslinker b),
20 to 95% by weight of vinylpyrrolidone and/or vinylcaprolactam,
0 to 40% by weight of at least one further monomer which is chosen from methyl(meth)acrylate, ethyl(meth)acrylate, n-butyl(meth)acrylate, C₈-C₂₂-(meth)acrylates, polyether(meth)acrylates terminated with C₈-C₂₂-alkyl groups, C₈-C₂₂-carboxylic acid vinyl esters and mixtures thereof.

20. The copolymer A) according to claim 18 which has a molar excess of cationogenic/cationic groups compared with anionogenic/anionic groups of at least 6:1.

21. The method as claimed in claim 1, wherein a copolymer is used, in which, for the copolymerization, two initiators are used whose decomposition temperatures are different from one another by at least 10° C.

22. The method according to claim 1, wherein the setting effect and/or the wet combability of the hair cosmetic composition is improved.

23. A hair cosmetic composition comprising
A) at least one ampholytic copolymer as defined in claim 1,
B) if appropriate at least one hair polymer different from A),
C) at least one cosmetically acceptable carrier, and
D) if appropriate at least one cosmetically acceptable active ingredient and/or auxiliary different from A) and B).

24. The composition according to claim 23 in the form of a setting foam, hair mousse, hair gel, hair wax, shampoo, hair spray, hair foam, end fluid, neutralizer for permanent waves or hot-oil treatment, in particular in the form of a hair gel, hair wax or shampoo.

25. A method of cosmetically treating the hair in which a composition as defined in claim 23 is prepared and applied to the hair to be treated.
